SEQUENCE LISTING (29 sequences)

SEQ ID NO: 1 - DNA, 55 bp, Artificial Sequence
  Synthetic construct
tggtgtggatccatatagatatcgtatcagctgctggacatttaggaccgtaccg

SEQ ID NO: 2 - DNA, 50 bp, Artificial Sequence
  Synthetic construct
cggtacggtcctaaatgtccagcagctgatacgatatctatatggatcca

SEQ ID NO: 3 - DNA, 29 bp, Artificial Sequence
  Synthetic construct
  misc_feature (7)..(7): n is a, c, g, or t
gtgccgnggtcctctgacgatatggaact

SEQ ID NO: 4 - DNA, 29 bp, Artificial Sequence
  Synthetic construct
cagttccatatcgtcagaggacccggcac

SEQ ID NO: 5 - DNA, 27 bp, Artificial Sequence
  Synthetic construct
  misc_feature (4)..(4): n is a, c, g, or t
gcgnggtcctctgacgatatggaactg

SEQ ID NO: 6 - DNA, 26 bp, Artificial Sequence
  Synthetic construct
cagttccatatcgtcagaggacccgc

SEQ ID NO: 7 - DNA, 26 bp, Artificial Sequence
  Synthetic construct
cagttccatatcgtcagaggacccgc

SEQ ID NO: 8 - DNA, 27 bp, Artificial Sequence
  Synthetic construct
  misc_feature (5)..(5): n is a, c, g, or t
gctgnggtcctctgacgatatggaact

SEQ ID NO: 9 - DNA, 27 bp, Artificial Sequence
  Synthetic construct
cagttccatatcgtcagaggacccagc

SEQ ID NO: 10 - DNA, 28 bp, Artificial Sequence
  Synthetic construct
  misc_feature (6)..(6): n is a, c, g, or t
gcctgnggtcctctgacgatatggaact

SEQ ID NO: 11 - DNA, 28 bp, Artificial Sequence
  Synthetic construct
cagttccatatcgtcagaggacccaggc

SEQ ID NO: 12 - DNA, 24 bp, Artificial Sequence
  Synthetic construct
tggtgtggatccatatagatatcg

SEQ ID NO: 13 - DNA, 44 bp, Artificial Sequence
  Synthetic construct
aaaaaaaaaattttttttttatcagctgctggacatttaggacc

SEQ ID NO: 14 - DNA, 38 bp, Artificial Sequence
  Synthetic construct
gtgccgtggtcctctgacgatatggaactggtccggga

SEQ ID NO: 15 - DNA, 33 bp, Artificial Sequence
  Synthetic construct
ggaccagttccatatcgtcagaggacccggcac

SEQ ID NO: 16 - DNA, 199 bp, Artificial Sequence
  Synthetic construct
attaatatatttattataaagaaatgtattgaatagattagtttagtatagaattgtaatgatttgatatgaatagtaaagtttagtttagaaatgtttgatttgtaaagtaaagtttagtattgatatgaaatgattagaattgtatagtaaagtaatgtattgaatagatttgattaatatatttattataaatccc

SEQ ID NO: 17-18 - skipped (2 entries)

SEQ ID NO: 19 - DNA, 29 bp, Artificial Sequence
  Synthetic construct
cctggtgagacctggctcctgacgatatt

SEQ ID NO: 20 - DNA, 29 bp, Artificial Sequence
  Synthetic construct
tttatcgtcaggagccaggtctcaccagg

SEQ ID NO: 21-24 - skipped (4 entries)

SEQ ID NO: 25 - DNA, 34 bp, Artificial Sequence
  Synthetic construct
  misc_feature (18)..(18): n is amino-C6-dT
  misc_feature (32)..(32): n is uracil
gtaccgctcctgacgttntcgtcaggagcggnac

SEQ ID NO: 26 - DNA, 56 bp, Artificial Sequence
  Synthetic construct
aacaaggtaccaacacaccacccacccaacccataatcaatctttcacctccaccc

SEQ ID NO: 27 - DNA, 56 bp, Artificial Sequence
  Synthetic construct
gggtggaggtgaaagattgattatgggttgggtgggtggtgtgttggtaccttgtt

SEQ ID NO: 28 - DNA, 42 bp, Artificial Sequence
  Synthetic construct
cggtacggtcctaaatgtccagcagctgatacgatatctata

SEQ ID NO: 29 - DNA, 43 bp, Artificial Sequence
  Synthetic construct
ttatagatatcgtatcagctgctggacatttaggaccgtaccg

(19) United States
(12) Patent Application Publication
(10) Pub. No.: US 2017/0218416 A1
(43) Pub. Date: Aug. 3, 2017

(54) COMPOSITIONS AND METHODS FOR SINGLE-MOLECULE CONSTRUCTION OF DNA

(71) Applicant: The Regents of the University of California, Oakland, CA (US)

(72) Inventors: Daniel H. Arlow, Berkeley, CA (US); William J. Holtz, Oakland, CA (US); Lane J. Weaver, San Francisco, CA (US); Jay D. Keasling, Berkeley, CA (US)

(73) Assignee: The Regents of the University of California, Oakland, CA (US)

(21) Appl. No.: 15/311,846

(22) PCT Filed: May 18, 2015
(86) PCT No.: PCT/US15/31444

§ 371 Date: Nov. 16, 2016

Related U.S. Application Data
(60) Provisional application No. 61/994,756, filed on May 16, 2014.

(51) Int. Cl. C12P 19/34; C12N 15/11

(52) U.S. Cl. CPC C12P 19/34; C12Y 207/07007; C12Y 605/01001; C12N 15/11; C12N 2310/531

(57) ABSTRACT

The present invention provides for compositions and methods for single-molecule construction of DNA. The present invention provides for a method comprising: (a) providing a reaction chamber comprising a solid support bound to a single starter double-stranded (ds) DNA molecule comprising a free end, (b) introducing one or more extension molecules and one or more enzymes capable of joining a payload region of an extension molecule to the free end of starter dsDNA molecule to the reaction chamber wherein the extension molecule comprises an cleavable linker.

### CROSS-REFERENCE TO RELATED APPLICATIONS

This application claims priority to U.S. Provisional Patent Application Ser. No. 61/994,756, filed on May 16, 2014, which is hereby incorporated by reference.

### STATEMENT OF GOVERNMENTAL SUPPORT

This invention was made with government support under Contract No. DE-AC02-05CH11231 awarded by the United States Department of Energy and Award No. EEC-0540879 awarded by the National Science Foundation. The government has certain rights in the invention.

### FIELD OF THE INVENTION

This invention relates generally to construction of DNA.

### BACKGROUND OF THE INVENTION

Construction of a DNA molecule with a desired sequence is a key step in nearly all life science research and bioengineering. Today, most DNA is still constructed by amplifying and modifying existing template sequences using molecular cloning techniques. Often, molecular cloning techniques fail for poorly understood reasons, resulting in lost time and missed research opportunities.

Increasingly, researchers are employing de novo “gene synthesis” services for construction of sequences for which no naturally occurring template exists/can be obtained, or for assistance in situations when traditional molecular cloning techniques fail. Most commercially available (or anticipated) gene synthesis techniques require the assembly of hundreds of ˜50 nucleotide (nt) synthetic oligonucleotides into kilobase-sized sequences, which are isolated, sequence-verified, and amplified for subsequent recombination into the full-length target sequence using molecular cloning techniques. Synthetic oligonucleotides are generated either via automated column synthesis at a cost of ˜$0.20/nt, or by massively parallel small-scale synthesis on microarrays at significantly reduced cost. The total gene synthesis cost and accuracy depend on the quantity and purity of synthetic oligonucleotides needed, the efficiency of the assembly technique, and the labor required for intermediate and/or final verification and error-correction. Currently, certain companies offering DNA synthesis, offers 3-5 kilobase (kbp) synthetic genes for $0.60/bp, with a turnaround time of 25 business days. This is both too slow and too expensive for most laboratories to use as a replacement for molecular cloning.

If it were possible to synthesize multi-kbp DNA with arbitrary sequence overnight for less than the labor and reagent costs of molecular cloning, it seems likely that most labs would stop all cloning work and would instead synthesize all of their DNA constructs, saving significant amounts of time and money. Furthermore, fast and inexpensive multi-kbp DNA synthesis would dramatically accelerate engineering in the life sciences, e.g. enabling rapid iteration in strain design and construction for metabolic engineering. Perhaps most significantly, if speed and cost improvements made multi-megabase (mbp) DNA synthesis accessible, it would enable previously impossible experiments such as full redesigns of microbial genomes, ushering in a new era of genome-scale research and engineering.

### SUMMARY OF THE INVENTION

The present invention provides for a method comprising: (a) providing a reaction chamber comprising a solid support bound to a single starter double-stranded (ds) DNA molecule comprising a free end, (b) introducing one or more extension molecules and one or more enzymes capable of joining a payload region of an extension molecule to the free end of starter dsDNA molecule to the reaction chamber wherein the extension molecule comprises an cleavable linker, (c) removing unligated extension molecules and the one or more enzymes from the reaction chamber, (d) determining that the payload region of the extension molecule is joined to the free end of the starter dsDNA molecule resulting in an elongation of the free end of the starter DNA molecule, (e) optionally repeating steps (b) to (d) until the elongation is determined, (f) cleaving the cleavable linker of the extension molecule, (g) removing the non-payload region of the cleaved extension molecule from the reaction chamber, (h) determining that the non-payload region of the cleaved extension molecule is cleaved and removed from the reaction chamber, (i) optionally repeating steps (f) and (g) until the non-payload region is removed from the reaction chamber, and (j) optionally performing one or more cycles of steps (b) to (h) such that each cycle results in the extension of the starter dsDNA molecule with one or more payload regions.

The present invention provides for a method comprising: (a) providing a reaction chamber comprising a solid support bound to a single starter double-stranded (ds) DNA molecule comprising a free end, wherein the free end is a blunt end and optionally a first label is linked to a 3′ end of negative strand of the single starter ds DNA molecule, (b) introducing one or more extension molecules and one or more enzymes capable of joining a payload region of an extension molecule to the free end of starter dsDNA molecule to the reaction chamber wherein the extension molecule comprises an cleavable linker, wherein the extension molecule comprises a hairpin loop and a second label, the cleavable linker comprises a deoxyuridine (dU) base, and optionally the payload region is one nucleotide, and wherein the first label and the second label are different labels that can be distinguished from each other, (c) removing unligated extension molecules and the one or more enzymes from the reaction chamber, (d) determining that the payload region of the extension molecule is joined to the free end of the starter dsDNA molecule resulting in an elongation of the free end of the starter DNA molecule, (e) optionally repeating steps (b) to (d) until the elongation is determined, (f) cleaving the cleavable linker of the extension molecule, (g) removing the non-payload region of the cleaved extension molecule from the reaction chamber such that a 3′ recessed end is created for the positive strand of the molecule bound to the solid support, (h) determining that the non-payload region of the cleaved extension molecule is cleaved and removed from the reaction chamber, (i) optionally repeating steps (f) and (g) until the non-payload region is removed from the reaction chamber, (j) filling in the 3′ recessed end to generate a blunt end for molecule bound to the solid support, and (k) optionally performing one or more cycles of steps (b) to (h) such that each cycle results in the extension of the starter dsDNA molecule with one or more payload regions. In some embodiments, the providing step (a) comprises filling in a 3′ recessed end of positive strand of the single starter ds DNA molecule to generate a blunt end.

In some embodiments, the filling in step comprises introducing a fill-in polymerase and dNTP to the reaction chamber. In some embodiments, a combination reaction solution comprising the fill-in polymerase and dNTP and cleavage reaction buffer is used for the filling in and cleaving steps. In some embodiments, the filling in step and the introducing one or more extension molecules steps are combined by combining the enzymes, dNTPs, and ExUs in a single reaction buffer.

In some embodiments, the (b) introducing step comprises: (i) introducing a DNA polymerase and dNTP, (ii) optionally removing the DNA polymerase and dNTP from the reaction chamber, and (iii) introducing an extension molecule and a ligase. In some embodiments, the (i) introducing step and the (iii) introducing step are separate.

In some embodiments, the (c) removing step comprises washing such that unjoined extension molecules and the one or more enzymes are removed from the reaction chamber. In some embodiments, the joining comprises ligating, and the enzyme capable of joining an extension molecule to the starter dsDNA molecule is a ligase, such as T4 ligase. In some embodiments, the cleavable linker of the extension molecule is a photo-cleavable linker, and the (f) cleaving step comprises irradiating the ligated extension molecule, such as irradiating with an ultraviolet (UV) light having a wavelength from 300 nm to 400 nm, or equal to or more than 365 nm. In some embodiments, the UV light has a wavelength of 300 nm to 370 nm. In some embodiments, the UV light has a wavelength of 300 nm to 350 nm. In some embodiments, the UV light has a wavelength of 360 nm to 370 nm. In some embodiments, the cleavable linker comprises a restriction site, and the (f) cleaving step comprises introducing an enzyme that cleaves the restriction site. In some embodiments, the enzyme is a restriction enzyme. In some embodiments, the cleavable linker comprises a chemical bond cleavable by a chemical capable of breaking the chemical bond, such as a reducing agent, such as tris(2-carboxyethyl)phosphine (TCEP).

The present invention provides for a composition comprising a reaction chamber comprising a single starter dsDNA molecule bound to a solid support.

The present invention provides for a composition useful as a starter dsDNA molecule in the method of the present invention comprising: (i) a starter solid support, and (ii) a + strand at least partially complementarily paired with a − strand, wherein both the + strand and the − strand each comprise a 5′ end and a 3′ end, optionally the − strand comprises an overhang of at least one nucleotide at the 5′ end of the − strand, and a PO₄ at the 5′ of the − strand, and the receptor is bound to the ligand, wherein the starter solid support is linked directly or indirectly to the 5′ end of the + strand. In some embodiments, the starter solid support is linked to a receptor which is in turn bound to a ligand which is linked to the 5′ end of the + strand. In some embodiments, the starter solid support is linked to the 5′ end of the + strand by a covalent bound. In some embodiments, the receptor is streptavidin, Traptavidin, or Neutravidin, and the ligand is biotin. In some embodiments, the + strand is fully complementarily paired with the − strand.

The present invention provides for a composition useful as an extension molecule (or extension unit) in the method of the present invention comprising: (i) a + strand at least partially complementarily paired with a − strand, wherein both the + strand and the − strand each comprise a 5′ end and a 3′ end or the + strand and the − strand form a hairpin loop, (ii) optionally a cleavable linker in a region where the + strand and the − strand form a duplex, or a cleavable linker in the − strand, at the 3′ end of the + strand or the 5′ end of the − strand, and (iii) optionally a PO₄, labelling compound or ligand linked to the 5′ end of the − strand, the 3′ end of the + strand, or a loop of the hairpin loop. The payload region comprises polynucleotide from the 3′ end of the − strand to the 5′ end of the − strand, or where cleavage takes place from the cleaving of the cleavable linker, or polynucleotides of the − strand which are designed or configured to be joined or ligated to the 5′ end of the − strand of the starter dsDNA molecule. The polynucleotides from the cleavable linker within the − strand to the 5′ end of the − strand form the constant region. In some embodiments, the extension molecule comprises (iv) one or more labelling compound wherein each labelling compound is linked to a nucleotide within the constant region.

In some embodiments, the extension molecule comprises: (i) a + strand at least partially complementarily paired with a − strand, wherein both the + strand and the − strand each comprise a 5′ end and a 3′ end or the + strand and the − strand form a hairpin loop, (ii) a cleavable linker in a region where the + strand and the − strand form a double strand, or a cleavable linker in the − strand, and (iii) optionally a labelling compound or ligand linked to the 5′ end of the − strand or a loop of the hairpin loop. In some embodiments, the extension molecule comprises (iv) one or more labelling compound wherein each labelling compound is linked to a nucleotide within the constant region.

In some embodiments, the extension molecule comprises: (i) a + strand at least partially complementarily paired with a − strand, wherein both the + strand and the − strand each comprise a 5′ end and a 3′ end or the + strand and the − strand form a hairpin loop, (ii) a cleavable linker in a region where the + strand and the − strand form a double strand, or a cleavable linker in the − strand, and (iii) a labelling compound or ligand linked to the 5′ end of the − strand or a loop of the hairpin loop.

In some embodiments, the extension molecule comprises: (i) a + strand at least partially complementarily paired with a − strand, wherein both the + strand and the − strand each comprise a 5′ end and a 3′ end, (ii) a cleavable linker in the − strand, and (iii) a labelling compound or ligand linked to the 5′ end of the − strand.

In some embodiments, the extension molecule comprises: (i) a + strand at least partially, of fully, complementarily paired with a − strand, wherein both the + strand and the − strand each comprise a 5′ end and a 3′ end or the + strand and the − strand form a hairpin loop, (ii) a cleavable linker in a region where the + strand and the − strand form a double strand, and (iii) a labelling compound or ligand linked to the 5′ end of the − strand or a loop of the hairpin loop. In some embodiments, the extension molecule comprises: (i) a + strand at least partially, of fully, complementarily paired with a − strand, wherein both the + strand and the − strand each comprise a 5′ end and a 3′ end, (ii) a cleavable linker in a region where the + strand and the − strand form a double strand, and (iii) a labelling compound or ligand linked to the 5′ end of the − strand. In some embodiments, the extension molecule comprises: (i) a + strand at least partially, of fully, complementarily paired with a − strand, wherein the + strand and the − strand form a hairpin loop, (ii) a cleavable linker in a region where the + strand and the − strand form a double strand, and (iii) a labelling compound or ligand linked to a loop of the hairpin loop. In some embodiments, the cleavable linker comprises a double-stranded (ds) DNA which is cleavable by an enzyme, such as a restriction enzyme. In some embodiments, the enzyme recognizes a recognition sequence in the dsDNA. In some embodiments, the enzyme cleaves the − strand at a location 3′ of the recognition sequence.

In some embodiments, the extension molecule comprises: (i) a + strand at least partially complementarily paired with a − strand, wherein both the + strand and the − strand each comprise a 5′ end and a 3′ end or the + strand and the − strand form a hairpin loop, (ii) a cleavable linker in a region where the + strand and the − strand form a double strand, or a cleavable linker in the − strand or at the 5′ end of the + strand, and (iii) one or more labelling compound wherein each labelling compound is linked to a nucleotide within the constant region. In some embodiments, the extension molecule comprises: (i) a + strand at least partially complementarily paired with a − strand, wherein both the + strand and the − strand each comprise a 5′ end and a 3′ end, (ii) a cleavable linker in a region where the + strand and the − strand form a double strand, or a cleavable linker in the − strand or at the 5′ end of the +strand, and (iii) one or more labelling compound wherein each labelling compound is linked to a nucleotide within the constant region. In some embodiments, the extension molecule comprises: (i) a + strand at least partially complementarily paired with a − strand, wherein the + strand and the − strand form a hairpin loop, (ii) a cleavable linker in a region where the + strand and the − strand form a double strand, or a cleavable linker in the − strand or at the 5′ end of the + strand, and (iii) one or more labelling compound wherein each labelling compound is linked to a nucleotide within the constant region.

In some embodiments, the extension molecule comprises: (i) a + strand at least partially, or fully, complementarily paired with a − strand, wherein both the + strand and the − strand each comprise a 5′ end and a 3′ end, (ii) optionally a cleavable linker at the 3′ end of the + strand or the 5′ end of the − strand, and (iii) a PO₄, labelling compound or ligand linked to the 5′ end of the − strand or the 3′ end of the + strand, wherein the payload region is the entire − strand. In some embodiments, the cleavable linker is located between the 5′ end of the − strand or the 3′ end of the + strand, and the labelling compound or ligand.

In some embodiments, the extension molecule comprises: (i) a + strand at least partially, or fully, complementarily paired with a − strand, wherein both the + strand and the − strand each comprise a 5′ end and a 3′ end, (ii) a PO₄, a cleavable linker, and a labelling compound or ligand linked, in this 3′ to 5′ sequence, to the 5′ end of the − strand, wherein the payload region is the entire − strand.

In some embodiments, the extension molecule comprises: (i) a + strand at least partially, or fully, complementarily paired with a − strand, wherein both the + strand and the − strand each comprise a 5′ end and a 3′ end, (ii) a PO₄ linked to the 5′ end of the − strand, and (iii) a cleavable linker, and a labelling compound or ligand linked, in this 5′ to 3′ sequence, to the 3′ end of the + strand, wherein the payload region is the entire − strand.

In some embodiments, the extension molecule comprises: (i) a + strand at least partially, or fully, complementarily paired with a − strand, wherein both the + strand and the − strand each comprise a 5′ end and a 3′ end, (ii) a PO₄ linked to the 5′ end of the − strand, and (iii) a labelling compound or ligand linked to the 3′ end of the + strand, wherein the payload region is the entire − strand.

In some embodiments, the extension molecule comprises: (i) a + strand at least partially, or fully, complementarily paired with a − strand, wherein both the + strand and the − strand each comprise a 5′ end and a 3′ end or the + strand and the − strand form a hairpin loop, (ii) a cleavable linker, comprising a dU, in a region where the + strand and the − strand form a duplex, or a cleavable linker in the − strand, and (iii) optionally a labelling compound or ligand linked to the 5′ end of the − strand, the 3′ end of the + strand, or a loop of the hairpin loop, wherein the + strand does not have a —OH its 3′ end. In some embodiments, when the + strand does not have a —OH its 3′ end, the 3′ end of the + strand comprises a dideoxy C terminus or a 3′ C3 spacer. In some embodiments, the payload region comprises polynucleotide at the 3′ end of the − strand, and optionally consisting of one nucleotide.

In some embodiments, the determining step (d), (h), or both, comprises detecting a single-molecule fluorescence through confocal microscopy or total-internal-reflectance microscopy. Detecting a single fluorescent DNA molecule, such as a single fluorescently-labeled DNA molecule on the surface of the reaction chamber, can comprise using a conventional confocal microscopy setup wherein a laser light (such as from a laser diode) from a pinhole source is reflected off a dichroic mirror through an objective and focused onto a spot containing the fluorescent DNA molecule. The fluorescent light emitted from the molecule is collected through the same objective, wherein the light is transmitted through a dichroic mirror (and optionally through another filter) and out-of-focus light is rejected by a pinhole aperture, and the light enters a photon-counting detector. Similar confocal fluorescence microscopy setups have been used successfully for detection of single molecules in other applications. When the single fluorescent DNA molecule is illuminated using total-internal-reflectance microscopy, the detecting is also by a photon-counting detector. In some embodiments, the photon-counting detector is an avalanche photodiode (APD) or a photomultiplier tube (PMT).

In some embodiments, the determining comprises introducing one or more stabilizing agents into reaction chamber or removing dissolved oxygen from the reaction chamber. Stabilizing agents enhance single-molecule fluorescence. When the fluorescent label for detecting the single DNA molecules is an organic fluorophore, such as Cy3 or Alexa 647, one can introduce one or more stabilizing agents to the reaction chamber during irradiation to prevent bleaching or blinking of the fluorophore during detection. In some embodiments, the reducing agent comprises (1) an antioxidant, such as 6-hydroxy-2,5,7,8-tetramethylchroman-2-carboxylic acid (Trolox), or (2) an oxygen scavenging system, such as a protocatechuic acid/protocatechuate-3,4-dioxygenase system. In some embodiments, removing dissolved oxygen from the reaction chamber comprises replacing the oxygen with an inert gas molecule, such as molecular nitrogen. In some embodiments, when the fluorescent label for detecting single DNA molecules is a quantum dot, the determining step comprises introducing a reducing agent, such as beta-mercaptoethanol, to reduce or suppress fluorescence intermittency or “blinking” of the fluorescent label.

In some embodiments, the cleavable linker is a photo-cleavable linker (such as the photo-cleavable linker shown in FIG. 2), a chemo-cleavable linker, or any cleavable structure that can be cleaved to reveal the payload region's 5′ PO₄. In some embodiments, the payload region comprises a 5′ phosphorothioate (PO₃S) with an alkyl linker off the sulfur to the 3′ end of the constant region. Treatment with AgNO₃ would convert the PO₃S into a standard 5′ PO₄ and would release the alkyl linker to the constant region.

In some embodiments, the payload region is about one to about ten nucleotides long. In some embodiments, the payload region has a length of one to about eight nucleotides. In some embodiments, the payload region has a length of about five nucleotides. In some embodiments, the constant region has a length of about ten to about fifty nucleotides. In some embodiments, the constant region has a length of about fifteen to about thirty nucleotides. In some embodiments, the constant region has a length of about twenty to about twenty-five nucleotides. In some embodiments, the constant region has a length of about twenty-three nucleotides. In some embodiments, the + strand has a length of about ten to about sixty nucleotides. In some embodiments, the + strand has a length of about twenty to about forty-five nucleotides. In some embodiments, the + strand has a length of about twenty-five to about thirty-five nucleotides. In some embodiments, the + strand has a length of about twenty-nine nucleotides.

In some embodiments, the payload region is about one to about 40,000 nucleotides long. In some embodiments, the payload region is about one, ten, 50, 100, or 500 to about 1,000, 2,000, 5,000, 10,000, 20,000, 30,000, or 40,000 nucleotides long. In some embodiments, the payload region has a number of nucleotides having a range from and to any of two numbers indicated above.

In some embodiments, the starter solid support is a surface of the reaction chamber, or a moveable surface, such as a magnetic bead. In some embodiments, the surface of the reaction chamber serves as a solid support for the growing starter molecule, or DNA molecule. In such embodiments, the surface or surfaces of the reaction chamber are passivated, such as passivated with a high-density polyethylene glycol brush, or carboxylate groups. In some embodiments, a small region of the surface or surfaces of the reaction chamber are chemically modified for immobilization of DNA in one or more of the following means: (1) addition of biotinyl groups for the binding of streptavidin, Traptavidin, or Neutravidin; (2) introduction of activated N-hydroxysuccinimide groups for conjugation with DNA containing a terminal amino group; (3) introduction of azides of conjugation with DNA containing a terminal alkyne via the Huisgen Azide-Alkyne 1,3-Dipolar Cycloaddition (i.e. “click” chemistry); or, (4) introduction of gold for binding of DNA containing a terminal thiol group. In some embodiments, the starter molecule, or DNA molecule, is bound to the starter solid support by one of the following means:

(1) Functionalize part of the reaction chamber surface with thiol groups (e.g. if it is glass, treat part with 3-mercaptopropyl trimethoxysilane), synthesize starter dsDNA with activated thiol group on the end (e.g. synthesizing the starter + strand 5′ end using the 1-O-dimethoxytrityl hexyl disulfide 1′[(2-cyanoethyl)-(N,N-diisopropyl)]phosphoramidite) and then immobilizing the starter dsDNA onto the surface via thiol/disulfide exchange at pH 9. This immobilization scheme has the advantage of being reversible; if a reducing agent like DTT is added to the reaction chamber, the disulfide is cleaved, liberating the DNA molecule and enabling reuse of the reaction chamber.

(2) Synthesize the starter dsDNA with a 3′ propanolamine group on the − strand, which will form a covalent adduct with glass in ddH2O at room temperature and neutral pH. This can be cleaved with strong acid or base.

(3) Any means taught in S. L. Beaucage, Curr. Med. Chem. 8:1213-1244 (2001).

In some embodiment, the extension unit comprises a ligand which is in turn bound to an extension solid support. In some embodiment, the extension solid support is smaller than the starter solid support. In some embodiment, the extension solid support is a bead, such as a magnetic bead, or quantum dot. In some embodiment, the labelling compound is a fluorophore. In some embodiments, the receptor molecule is a protein molecule. In some embodiments, the ligand molecule is a protein molecule. In some embodiments, the receptor molecule is streptavidin, and the ligand molecule is biotin. In some embodiments, the receptor molecule is a Fab fragment of an antibody, and the ligand molecule is an antigen or hapten.

In some embodiments, the receptor molecule binds the ligand molecule with an association constant (Kₐ) equal to or more than about 10⁸ mol/L. In some embodiments, the association constant is equal to or more than about 10¹⁰, 10¹¹, 10¹², 10¹³, or 10¹⁴ mol/L. In some embodiments, the receptor molecule and the ligand molecule when bound together have a dissociation rate of about 2.4×10⁻⁶ s⁻¹. In some embodiments, the dissociation rate is equal to or less than about 2.0×10⁻⁶ s⁻¹, 1.0×10⁻⁶ s⁻¹, 2.0×10⁻⁷ s⁻¹, or 2.0×10⁻⁸ s⁻¹.

The present invention provides a device configured to comprise a reaction chamber in fluid communication to one or more storage chambers comprising one or reagents for the practice of the method of the present invention, and one or more input ports wherein one or reagents are introduced to the device wherein each reagent is introduced through one or more input port. In some embodiments, the device comprises a separate input port for each reagent that is introduced. In some embodiments, the device comprises one or more of the features shown in FIG. 4.

The present invention provides a method for rapid construction of multi-kilobase arbitrary-sequence DNA by sequential enzymatic extension of a single DNA molecule on solid support by capped double-stranded DNA “extension units”. Unlike bulk synthesis, this method for constructing exactly one molecule of DNA enables monitoring the completion of each extension step to ensure production of a correct full-length final product. Furthermore, this method dramatically reduces reagent costs and enables certain speedups over bulk reactions. Once a single molecule with the desired sequence has been constructed, it can be amplified for subsequent molecular biology applications.

The present invention provides for a method for constructing multi-kbp synthetic DNA molecules by sequential assembly of addition units carrying a short DNA “payload region” to a single growing DNA molecule on solid support. In some embodiments, an appropriate implementation of the method enables essentially error-free synthesis of arbitrary sequence multi-kbp DNA overnight. The present invention enables DNA construction at a cost far less than using current methods of molecular cloning, and is an enabling technology for genome-scale DNA synthesis.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 shows a single-molecule DNA assembly process.

FIG. 2 shows a standard design of a dsDNA extension unit.

FIG. 3 shows a reaction cycle with standard extension units.

FIG. 4 shows integrated microfluidic devices for single-molecule DNA construction. Panel (A): A representation of a chip implementing the reaction cycle using electrowetting-on-dielectric. Panel (B): Integrated microfluidic device for single-molecule DNA construction. The device comprises of micro-channels and computer-controlled micro-valves made of PDMS bonded to a coverslip.

FIG. 5-1 shows an alternate internally cleavable dsDNA extension unit.

FIG. 5-2 shows alternate fluorescent labeling strategies for internally cleavable dsDNA extensions.

FIG. 6 shows extension unit designs with terminal caps.

FIG. 7 shows a reaction cycle for ex. Units with non-cleavable caps.

FIG. 8 shows bulk ligation of extension units with photocleavable linkers to payload regions of 3-6 nucleotides.

FIG. 9 shows bulk sequential ligation of PC-Spacer 6-mer extension units.

FIG. 10 shows a reaction chamber is loaded with a single phosphorylated starter DNA molecule that is 3′ recessed by 1 nt. B. The molecule is filled in until blunt by a polymerase. C. Extension units (ExUs) carrying the next 1-bp payload are introduced to the reaction chamber. T4 ligase joins the starter's 5′ PO₄ to one ExU via the 3′ OH of its 1-nt payload (orange), leaving a nick on the opposite strand. D. The reaction chamber is flushed to remove free extension units and is imaged for single-molecule fluorescence. If no fluorescence is detected, the extension step is reattempted. E. The deprotection reagent is introduced to the reaction chamber, which cleaves the backbone at the dU base immediately adjacent to the delivered payload, releasing the fluorophore-conjugated “constant region” of the ExU into solution, and exposing the 5′ PO4 of growing DNA molecule for subsequent extension. F. The reaction chamber is flushed to remove the deprotection product and is imaged for single-molecule fluorescence. If fluorescence is detected, the deprotection step is reattempted. Otherwise, the reaction cycle can now repeat with the next 1-bp extension. After the desired molecule has been synthesized, it is amplified to yield a useable quantity of DNA.

FIG. 11 shows embodiments of extension units. Panel (A) shows “iCy3 dU:A GT hairpin”: 5′-GTAccgctcctgacgTTXTcgtcaggagcggUAC-3′ (SEQ ID NO:25) where X is amino-C6-dT (see IDT catalog, Integrated DNA Technologies Inc., Coralville, Iowa) coupled to NHS-sulfo-Cy3 (such as commercially available from Lumiprobe Corp., Hallandale Beach, FA). This ExU will be referred to as “iCy3 GT ExU” for brevity in the experiments described below. Panel (B) shows a 1 nt payload ExU with modified nucleobase as cleavable linker. Panel (C) shows a ExU (similarly to that shown in panel (B)) with multiple fluorophores covalently attached to modified bases in the constant region. Multiple redundant fluorophores reduce the risk that photobleaching (or chemical bleaching) results in a completely non-fluorescent ExU. Multiple fluorophores can be conjugated an ExU containing multiple amino-dT bases via NHS coupling.

FIG. 12 shows an iteration of the reaction cycle in solution. Panel (A) shows a 56 nt 5′ phosphorylated dsDNA molecule labeled with 5′ TET and 3′ FAM serves as a “starter DNA molecule” (starter) that is iteratively extended by 2 bp donated by the iCy3 GT ExU in the reaction cycle. The starter is ligated with an excess of ExU in solution and purified by PCR Cleanup, (resulting in removal of 99.9% of the ExU due to length-dependence of retention,) producing nicked dsDNA labeled with FAM, TET, and Cy3. Subsequently, the DNA is “deprotected” with the USER Enzyme, leaving a 5′ PO₄ and a 3′ end recessed by 2 nt; without further purification, Klenow polymerase and dNTPs are added to the reaction mixture to fill-in the 3′ end until blunt, preparing the extended starter DNA molecule for subsequent extension. Panel (B) shows a gel-like image generated from C.E. chromatograms of samples of purified DNA taken at each step of 2 iterations of the reaction cycle, demonstrating that the reaction cycle extends both strands of the starter by 2 nt per cycle. C.E. chromatograms are resealed so that the maximum signal from the FAM channel had equal intensity in all samples. (Note that even though both starter strands are initially 56 nt, their distinct labels result in different electrophoretic mobility and thus non-overlapping FAM and TET bands.)

FIG. 13 shows distinctive dynamical behavior of ligated vs. nonspecifically bound ExUs. Panel (A) shows the result of a successful ligation step, the ExU (orange) is ligated to the growing DNA molecule (blue) on the reaction chamber surface. Panel (B) shows the ExU has nonspecifically absorbed to the reaction chamber surface near the site of the growing DNA molecule. Panels (C) and (D) show the hypothetical diffusive trajectories of the fluorophores attached to the ExU from Panels (A) and (B), respectively. Panels (E) and (F) show the simulated single-molecule fluorescence images integrated over the ExU trajectories depicted in Panels (C) and (D), respectively. In these hypothetical images, it is clear that the ligated ExU has detectibly different spatial fluctuations than the nonspecifically bound ExU.

FIG. 14 shows a single-molecule loading strategy.

FIG. 15 shows a post-synthesis single-molecule isothermal linear amplification scheme. Panel (A) shows Nt.BbvCI nicks the single DNA molecule at the part of the sequence corresponding to the end of the hybridization tag used initially to immobilize the “starter DNA molecule” (see FIG. 14), effectively creating a primer-template junction for the polymerase to initiate elongation. Panel (B) shows that as the polymerase proceeds with elongation, it displaces the + strand of the template into solution. Panel (C) shows the nicking endonuclease can regenerate the nick at the 5′ end of the template, creating another primer-template junction from which the polymerase can initiate.

FIG. 16 shows results for tracking a single starter DNA molecule through three iterations of the reaction cycle. Panel (A) shows the Alexa Fluor 647-labeled starter DNA and Cy3-labeled extension unit (ExU) used for this demonstration. Panel (B) shows an image of Cy5-channel fluorescence of the sample chamber after loading with starter DNA; insets: magnified view of Cy5- and Cy3-channel fluorescence of a small region of the sample chamber centered on one particular starter DNA molecule (indicated by the arrow). Panel (C) shows images of Cy3-channel fluorescence of the highlighted region of the reaction chamber taken after loading the reaction chamber with starter DNA (“S”), the first ligation (“L1”), deprotection (“D1”), and fill-in (“F1”) steps, and continuing on to the second (“L2”, “D2”, “F2”) and third iterations (“L3”, “D3”, “F3”) of the complete reaction cycle. The appearance of the central spot during each ligation step and its subsequent disappearance in each deprotection step indicates that the central starter DNA molecule has completed three iterations of the reaction cycle.

### DETAILED DESCRIPTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a molecule” includes a plurality of such molecules, and so forth.

The term “about” refers to a value including 10% more than the stated value and 10% less than the stated value. When used to describe a number of nucleotides, it also includes a number of nucleotides one more and/or one fewer than the stated number.

The term “strand” refers to a polynucleotide, such as a single-stranded DNA.

These and other objects, advantages, and features of the invention will become apparent to those persons skilled in the art upon reading the details of the invention as more fully described below.

In one aspect of the invention, each reaction cycle iteratively extends a single double-stranded DNA molecule on solid support by one “capped” double-stranded DNA extension unit, leaving the extended molecule in a state suitable for a subsequent extension reaction. In some embodiments, each extension unit is directionally “capped” to prevent multiple extensions from occurring. In some embodiments, each reaction cycle comprises one or more of the following four steps: (1) introducing an extension unit and DNA ligase to the reaction chamber, (2) washing to remove of unreacted capped extension units, (3) deprotecting the growing DNA molecule (i.e., removing the “cap”), and (4) washing to remove soluble deprotection products.

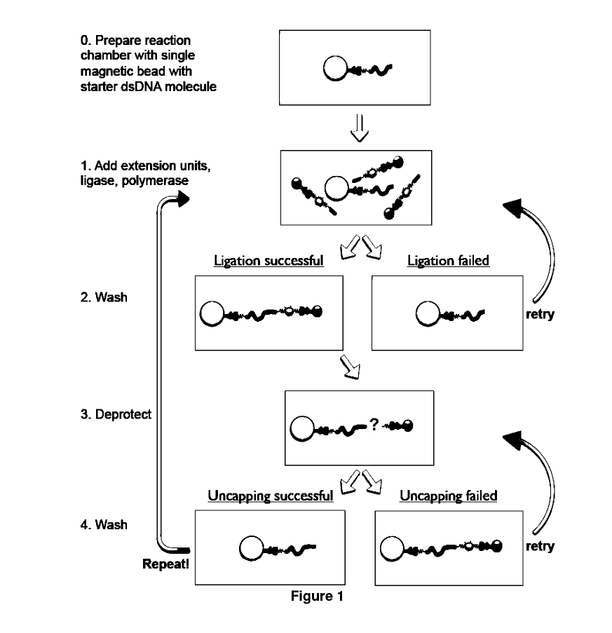

In one aspect of the invention, when constructing a single molecule, the reaction scheme permits monitoring the completion of each addition step, one can wait until the reaction happens before proceeding to the next step, thereby ensuring error-free construction of the full-length molecule (FIG. 1). The invention avoids one of the problems of prior art methods, which is the geometric reduction in yield for sequential bulk reactions as the number of cycles increases.

In one aspect of the invention comprises biochemical implementation using blunt-end ligation of fluorophore or quantum dot-conjugated double-stranded DNA units with an internal photocleavable linker

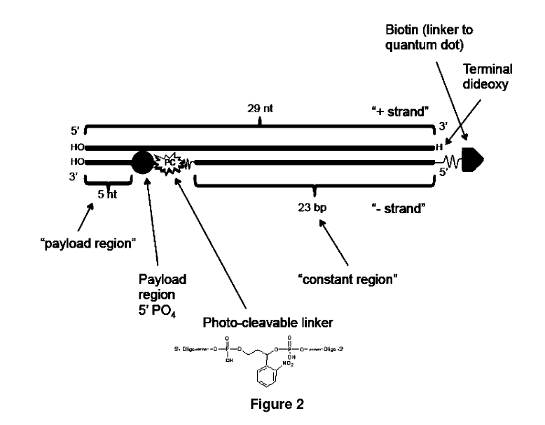

Extension unit design. In some embodiments, extension molecule comprises a “standard design” (FIG. 2) comprising a duplex DNA comprising two strands: a “− strand” and a “+ strand”. In some embodiments, the − strand comprises an oligonucleotide with its 5′ end conjugated to a ligand, such as a biotin molecule, followed by a “constant region”, followed by a cleavable linker, such as a 2-nitrobenzyl photocleavable linker, connecting the 3′ phosphate of the constant region to the 5′ phosphate of the “payload region”. The payload region is designed or configured to be joined, ligated or appended to the growing DNA molecule in the method of the present invention. In some embodiments, the + strand is a standard oligonucleotide with a 3′ dideoxy nucleotide complementary to the − strand with an additional nucleotide (or appropriate spacer group) in the position complementary to the cleavable linker. In some embodiments, the hybridized extension unit comprises a DNA duplex comprising one blunt end exposing a 3′ hydroxyl and a 5′ hydroxyl and the other end exposing a ligand at the 5′ end and no 3′ hydroxyl. In some embodiments, the 5′ biotin group of the − strand is used to bind a receptor-conjugated quantum dot that is used to track ligations and photocleavage of the linker. In some embodiments, a fluorescent dye such as Cy5 is covalently attached to the − strand instead of a receptor that is useful for tracking. In some embodiments, the − strand is attached a chemical moiety that permits single molecule detection, such as a nanoparticle that noticeably affects the capacitance or resistance of the reaction chamber. In some embodiments, the extension molecule is compatible with blunt-end ligation of the − strand 3′ hydroxyl to the 5′ phosphate of the growing DNA molecule in the method of the present invention. In some embodiments, the ligation is catalyzed by a ligase, such as T4 DNA ligase. In some embodiments, the ligation joins a quantum dot fluorophore (via a biotin-streptavidin linkage), through the constant region of the extension unit, through the cleavable linker, through the payload region, to the growing DNA molecule on solid support, while leaving a nick on the + strand at the ligation junction. Since the extension units are not phosphorylated on their exposed 5′ termini, they will not ligate to each other. In some embodiments, the constant region can serve a spacer separating the bulky quantum dot from active site of the ligase, preventing interference. Strong hybridization of the constant region of the − strand to its complement on the + strand constrains and orients the − strand's payload region alongside its complementary sequence on the + strand so that the extension unit can present blunt duplex DNA to the ligase even though complementary oligonucleotides as short as the payload region would be predominantly dissociated at room temperature.

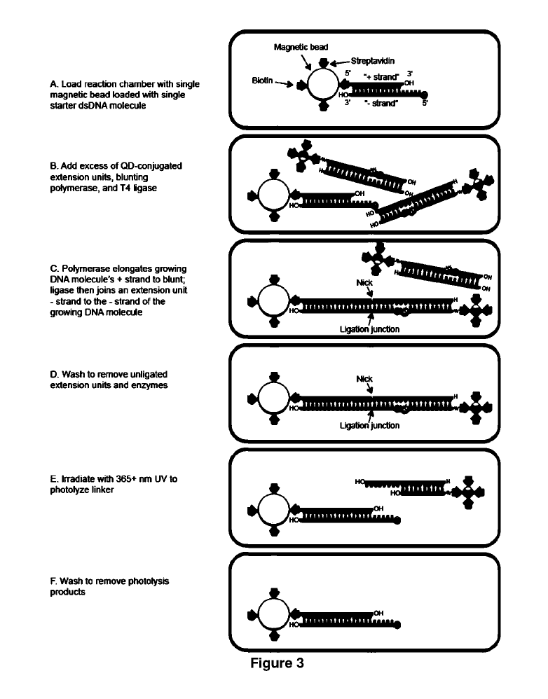

Reaction cycle. In some embodiments, the invention comprises (as depicted in FIG. 3) a solid support (such as a single streptavidin-coated magnetic bead) prepared with a single “starter” DNA molecule consisting of two strands, named the “− strand” and the “+ strand” as above. The “− strand” is a 5′ phosphorylated oligonucleotide containing a universal primer binding site for subsequent polymerase chain reaction (PCR) amplification of the assembled DNA molecule; the “+ strand” is an oligonucleotide complementary to the “− strand” with a 5′ biotin group for immobilizing the starter DNA molecule onto the solid support, and may optionally be recessed by a few bases on its 3′ end. In some embodiments, the assembled DNA molecule is amplified through nicking enzyme amplification reaction (NEAR), wherein hybridizing primers are not required/used. A nicking enzyme is used to create nick(s) near the 5′ end of the template, and each nick then serves as a primer junction for amplification. NEAR is a method for in vitro DNA amplification. NEAR is isothermal, replicating DNA at a constant temperature using a polymerase (and nicking enzyme) to exponentially amplify the DNA at a temperature range of 55 ° C. to 59 ° C. NEAR is disclosed by U.S. Pat. No. 7,972,820. The single starter DNA on solid support is placed in the reaction chamber (FIG. 3A). In some embodiments, the reaction cycle comprises : (1) an excess of (fluorophore-conjugated) extension unit stock is added to the reaction chamber, together with blunting polymerase (e.g. Klenow or T4 Polymerase), T4 ligase, and reaction buffer containing ATP and dNTPs (FIG. 3B). Blunt-end ligation of an extension unit to the growing DNA molecule proceeds immediately following elongation of the + strand to blunt by the polymerase, leaving a nick on the + strand at the ligation junction (FIG. 3C). (2) After an incubation period, the reaction chamber is washed with Wash Buffer to remove all free extension units (FIG. 3D). (3) Fluorescence of the reaction chamber is measured. If the fluorescence of a single extension unit is detected, it is concluded that the ligation has succeeded in joining the extension unit to the growing DNA molecule on solid support, and the reaction cycle can proceed. Otherwise, it is concluded that the ligation has failed, and is reattempted (i.e. go to step 1). (4) The reaction chamber is irradiated with 330+ nm UV light for a set period of time to photolyze the photocleavable linker in the extension unit, thus leaving an unprotected 5′ phosphate on the growing DNA molecule (FIG. 3E). Since the + strand contains a nick at the ligation junction, the region of the + strand 3′ of the ligation junction (i.e. complementary to the payload region) dissociates from the growing DNA molecule. (5) The reaction chamber is washed with Wash Buffer to remove the photolysis products (FIG. 3F). (6) Fluorescence of the reaction chamber is again measured. If the fluorescence of a single extension unit is detected, it is concluded that the photolysis has failed, and is reattempted (i.e. go to step 4). Otherwise, it is concluded that the photolysis has succeeded, and the reaction cycle can proceed through another addition (i.e. go to step 1).

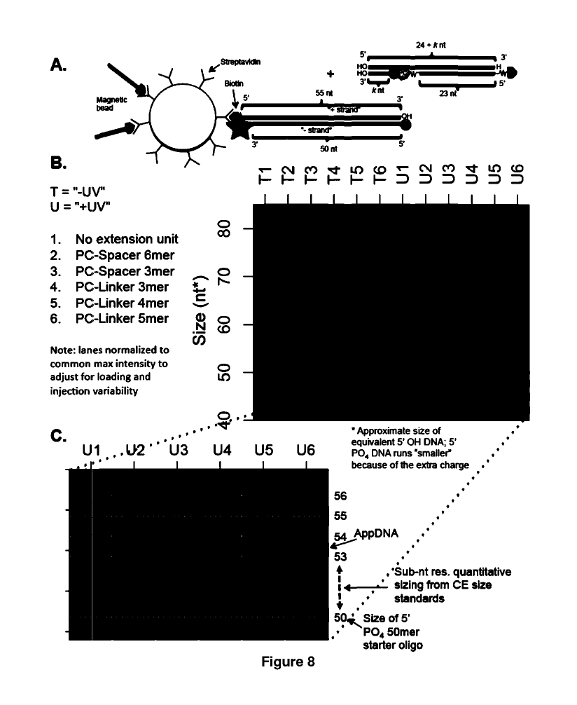

In some embodiments, the invention also comprises a reaction cycle shown in FIG. 10. Panel A shows the reaction chamber is loaded with a single phosphorylated starter DNA molecule that is 3′ recessed by 1 nt. Panel B shows the molecule is filled in until blunt by a polymerase. Panel C shows the extension units (ExUs) carrying the next 1-bp payload are introduced to the reaction chamber. T4 ligase joins the starter's 5′ PO₄ to one ExU via the 3′ OH of its 1-nt payload (orange), leaving a nick on the opposite strand. Panel D shows the reaction chamber is flushed to remove free extension units and is imaged for single-molecule fluorescence. If no fluorescence is detected, the extension step is reattempted. Panel E shows the deprotection reagent is introduced to the reaction chamber, which cleaves the backbone at the dU base immediately adjacent to the delivered payload, releasing the fluorophore-conjugated “constant region” of the ExU into solution, and exposing the 5′ PO4 of growing DNA molecule for subsequent extension. Panel F shows the reaction chamber is flushed to remove the deprotection product and is imaged for single-molecule fluorescence. If fluorescence is detected, the deprotection step is reattempted. Otherwise, the reaction cycle can now repeat with the next 1-bp extension. After the desired molecule has been synthesized, it is amplified to yield a useable quantity of DNA (not shown).

In some embodiments, a final extension unit that is added to the growing chain is a special extension unit that has a universal (reverse) primer binding site in its payload region. Addition of this unit to completed DNA molecule on solid support thus brackets the constructed sequence with universal primer binding sites, enabling amplification. (Alternately, primer binding sites may be synthesized directly into the sequence using standard addition units.) The final step of construction is amplification of the single constructed DNA molecule using the universal primers, resulting in many soluble, unmodified copies of the constructed DNA molecule suitable downstream applications.

The invention has one or more of the following advantages of single-molecule blunt- end assembly over cohesive end bulk assembly.

Advantages of blunt-end ligation over cohesive-end ligation. In standard molecular biology bench work, ligation of DNA molecules with overhangs is typically preferred over blunt-end ligation because use of distinct overhang sequences allows for directional cloning. In one aspect of the present invention, directionality is achieved in blunt-end ligation by chemically protecting the termini of all DNA molecules such that undesirable ligations are infeasible: the growing chain is conjugated to the solid support via biotinylation of the 5′ end of it's + strand, and the extension units have a dideoxy base at the 3′ end of their − strand, are conjugated to biotin (or an organic fluorophore) via the 5′ end of their − strand, and are not phosphorylated on the 5′ end of their + strand. Thus, the only phosphorylated 5′ end available during the ligation step is the 5′ end of the growing chain's − strand, and the only 3′ hydroxyls available for ligation are on the end of the extension unit payload region.

There is a misconception that ligation of DNA with overhangs proceeds at a faster rate due to the existence of a population of molecules in which the overhangs are hybridized. However, the assumption that this should lead to a significant speedup in ligation is questionable given that the predicted melting temperature of overhangs consisting of 2-4 bases is well below room temperature under the conditions in which ligations are typically preformed. The product information for the M2200 Quick Ligation Kit on the New England Biolabs website shows that ligation of Phage Lambda DNA digested with a restriction enzyme that leaves a blunt ends proceeds at more than half the speed of ligation on the same DNA digested with a restriction enzyme that leaves a three-base overhang. Under suitable conditions, it should be possible to achieve ligation of an extension unit to the growing DNA molecule with high probability in under a minute. Directionally-capped blunt-end ligation offers several key advantages over cohesive-end ligation for DNA assembly. Cohesive-end ligation requires complementarity between the ends of the DNA molecules to be joined, necessitating constraints on the terminal sequences of both DNA molecules. As such, the size of the library required to cover all extension units that add k new nucleotides onto DNA with a j-base overhang is 4^{k+j}—potentially much greater than the 4^{k} library necessary for covering all extension units of length k. For example, to add three nucleotides with a 3-base overhang would require a library with 4⁶=4096 extension units—much more than the 4³ extension unit library required for blunt-end ligation.

Decreased average cycle duration from single-molecule tracking. In some embodiments, in addition to ensuring 100% accurate construction, the single-molecule scheme can achieve significantly shorter reaction cycle times compared to any bulk coupling scheme by strategically monitoring the reaction progress. If the ligation reaction proceeds at a rate λ; in the bulk scheme, this means that the fraction of extended molecules as a function of time t will increase as 1−e^{-λt}, and in the single-molecule scheme this means that the probability that the growing chain is extended in t seconds is given by the same equation. In the bulk scheme, the ligation reaction must be run for substantially longer than the time constant 1/λ to guarantee high yields; for example, running the reaction for 3/λ seconds is necessary to achieve 95% yield, and 4/λ seconds is necessary to achieve 98% yield. However, in the single-molecule scheme, ligation failures are detected and the ligation step is reattempted, leading to 100% yield. If it is incubated for x seconds and then wash for w seconds and finally measure fluorescence (for a negligible amount of time). In this case, the time until the ligation step is successfully completed is (x+w)N where N is a geometrically distributed random variable with the parameter θ=1−e^{-λx}, corresponding to the probability of ligation in x seconds. The expected value of this expression is thus (x+w)/θ=(w+w)/(1−e^{-λx}), which one can minimize with respect to x in order to optimize the duration of the ligation step. For example, if w=30 seconds and 1/λ=60 seconds, then the optimal x is roughly 141 seconds and results in a expected waiting time of ˜189 seconds, of which 30 seconds is the final wash. For comparison, if a bulk reaction with the same parameters were carried out for 189−30=159 seconds, only 93% of molecules would be extended.

Optionally, it is possible to immediately detect ligation and/or deprotection by detecting changes in fluorophore localization or dynamics Upon ligation, the fluorophore becomes tethered to the solid support, significantly slowing its diffusion and confining it to a small region of the reaction chamber; during deprotection, the situation is reversed. As such, certain optical setups may allow for immediate detection of the ligation through changes in the diffusive motion of the fluorophore. This would be advantageous both for accelerating the reaction cycle and for limiting the UV exposure of the growing DNA molecule and fluorophore during deprotection.

Microfluidic integration using electrowetting-on-dielectric. Efficient and rapid dispensing and transport of >4⁵ reagents, (including the library of directionally-capped extension units of all possible sequences,) is required for successful multi-kilobase DNA synthesis in a day. Reactions can be performed in sub-μL volumes. Microfluidic integration using electrowetting-on-dielectric technology (a.k.a. “digital microfluidics”) is ideally suited to the requirements of a device for automating the reaction scheme described above. Digital microfluidics devices can reliably dispense droplets as small a picoliter from an on-chip reservoir and transport them around the chip. Furthermore, use of an immiscible filler medium such as silicone oil mitigates nonspecific absorption of DNA onto chip surfaces, reducing carryover. The design described herein is robust to trace enzyme carryover provided that washing strictly removes DNA, and as such, is ideally suited to digital microfluidics implementation.

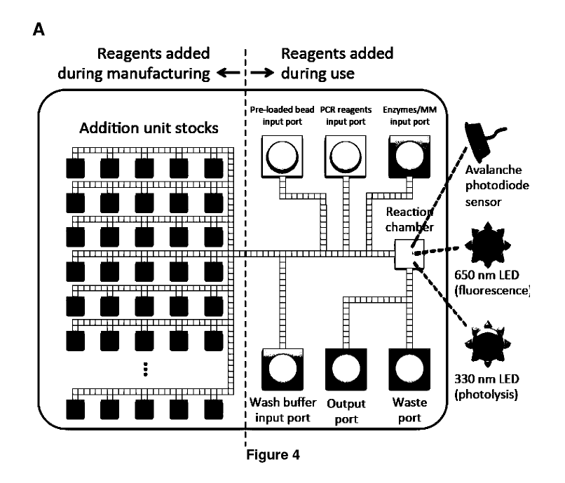

A device implementing the reaction scheme described herein is depicted in FIG. 4. Half of the device comprises ports and reservoirs for wash buffers, enzymes, waste, and constructed DNA products, and a special region that functions as the reaction chamber. The other half of the device comprises 4^{k} reservoirs of the extension unit stocks for all payload region sequences of length k. and a bus that connects each reservoir to the reaction chamber area. In one aspect of the invention, the device comprises 4^{k} extension units pre-loaded on the chip (during manufacturing), and is optionally a disposable cartridge. Mounted above the reaction chamber are the optics for detecting single-molecule fluorescence, such as a red LED for excitation, an avalanche photodiode (with an implied far-red filter) for detection of single-molecule fluorescent emission, and/or a long-wavelength UV LED for deprotection. The device can further comprise a computer which through a controller applies voltage to electrode pads to actuate droplet movement within the channels of the device. Software on an attached computer can take as input a DNA sequence to be constructed, partition it into length k subsequences, and implement the construction scheme depicted in FIG. 1 followed by (optionally on-chip amplification and elution to the output port.

Adding multiple independent reaction chambers to the chip enables parallel construction of several target DNA molecules. Notably, for k=5, the chip already has 1024 reagent stocks, so adding 1000 reaction parallel chambers only double the microfluidic complexity while enabling 1000 times greater DNA construction throughput. Such a single-reaction-chamber device, can synthesize over a megabase of DNA within a day.

While electrowetting-on-dialectric microfluidics are ideally suited to the requirements of this reaction scheme, alternate microfluidic technologies such as pneumatically-actuated PDMS-on-glass “pipes and valves” can be substituted. Optional designs also include a disposable mobile solid support, or even immobilize the growing DNA molecule on a functionalized surface of the device itself.

QDot 705 Streptavidin conjugates are commercially available from Invitrogen (Carlsbad, Calif.). The method can be practiced using about 100 μmol of extension units in an about 20 μL working volume; scaling this down to (relatively large) about 1 nL droplets means requiring about 5 fmol of quantum dots per attempt. The droplet volume can also be reduced to about 10 pL. Quantum dots for single molecule tracking can be replaced with a fluorescent dye such as CyS.

### Other Biochemical Implementations of the Invention

Universal 5′ overhang on the + strand for pseudo-cohesive-end ligation. In some embodiments, it is possible to accelerate the reaction cycle beyond the maximum speed achievable with blunt-end ligation by switching to cohesive-end ligation. This is accomplished without suffering from the disadvantages of cohesive-end ligation described above through the use of universal bases (such as 5-nitroindole) that can base-pair with any standard base. In this scheme, the 5′ end of the + strand of the extension unit is extended by several universal bases to produce a universal overhang that can hybridize with any template sequence. If the end of the growing DNA molecule on solid support has not been blunted, hybridization with an extension unit's universal overhang will stabilize and orient the DNA complex for ligation, possibly accelerating the reaction.

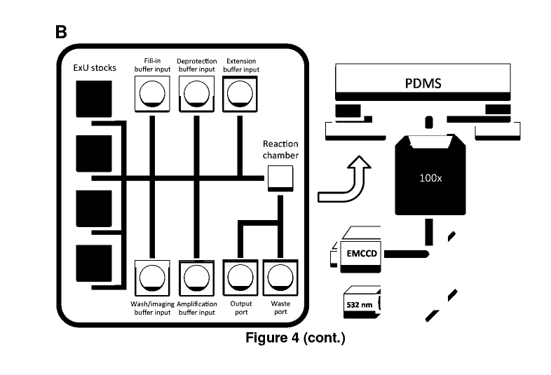

Alternate cleavable linkers for the − strand. In some embodiments, an alternative to long-wave UV irradiation for deprotection is the use of other internal cleavable linker groups (FIG. 5) substituted in the extension unit design, such as photocleavable linkers that absorb in the visible region of the spectrum, or chemically cleavable linkers that can be rapidly cleaved upon addition of some reagent, e.g. a disulfide cleaved by a reducing agent such as TCEP (FIG. 5A). (In some embodiments, this substitution may be necessary if irradiation required for photolysis of the 2-nitrobenzyl linker inevitably damages DNA.) The essential properties of the cleavable linker are that it: (1) stably links the constant region of the extension unit − strand's payload region to its constant region, (2) is rapidly cleavable under conditions compatible with the reaction scheme above, (3) is compatible with the conditions for ligation of the extension unit to the growing chain by T4 ligase and elongation of the + strand by the blunting polymerase, and (4) upon cleavage reveals the 5′ phosphate of payload region of the − strand. This also includes linkers that are not covalently bonded to the 5′ phosphate of the payload region but instead are bonded to the payload region via some other covalent linkage, e.g. via an O-allyl or disulfide linkage to a free sugar hydroxyl. In some embodiments, double-stranded (FIG. 5B) or hairpin (FIG. 5C) extension units contain a type-IIs (offset cutting) or nicking endonuclease site which fulfills these criteria, so long as the constant region has the recognition sequence oriented in a way that the cut site on the − strand occurs at the 5′ end of the payload region and the enzyme either doesn't cut on the + strand or the cut site is located after the ligation junction.

A disadvantage of designing an endonuclease-cleavable extension unit is that the recognition sequence of the endonuclease is prohibited from occurring in the sequence to be synthesized; otherwise, during a deprotection step the growing DNA molecule could be cut at the internal synthesized recognition site. However, this disadvantage can be mitigated by chemically modifying the payload region of all (relevant) extension units in such a way that any incidentally synthesized endonuclease recognition sites in the growing DNA molecule will not permit cleavage by the endonuclease. In these embodiments, the − strand of the growing DNA molecule comprises entirely of payload regions. Oligonucleotides with such chemical modification(s) are to be compatible with ligation by T4 ligase.)

A specific example of such a chemical modification is methylation of the C5 position of cytosine, which will block recognition and cleavage by Bsal, a type-II endonuclease. In some embodiments, a hairpin extension unit with a 5-base payload (5′ CCAGG 3′ (SEQ ID NO:17), shown in bold) that is Bsal-cleavable (the Bsal recognition 5′ GGTCR 3′ (SEQ ID NO:18) is shown in bold italics; but the enzyme cuts at the +1 position 3′ of the recognition site on that strand and at the +5 position on the opposite strand).

| | |
|---|---|
| + strand: | 5′ CCTGGt [symbol] tggctcctgacgatatt (SEQ ID NO: 19) |
| | \|\|\|\|\|\|\|\|\|\|\|\|\|\|\|\|\|\|\|\|\|\|\|\|\|\|\|\| }~Cy3 |
| - strand: | 3′ GGACCa [symbol] accgaggactgctattt (SEQ ID NO: 20) |

In such an embodiment, the payload region begins with 5′ CC 3′, which could form the beginning of an incidentally synthesized Bsal site (5′ GAGACC 3′ (SEQ ID NO:21))—for example if the next payload region(s) were 5′ AGAGA 3′ (SEQ ID NO:22), then the sequence of the − strand begins with 5′ AGAGACCAGG 3′ (SEQ ID NO:23), which contains a BsaI recognition site 5′ GAGACC 3′. By synthesizing the extension unit to contain a C5-methylcytosine at the position indicated by the “°” symbol above, the payload is prevented from becoming part of an incidentally synthesized BsaI recognition site. In this specific embodiment, 5′ AGAGACCAGG 3′ is not recognized by Bsal since the “C” indicated by underline is C5 methylated.

In some embodiments, the dNTPs added during the fill and ligation step(s) are replaced with chemically modified dNTPs such that the nucleotides incorporated into the + strand by the polymerase are chemically modified so that cleavage is blocked at any incidentally synthesized endonuclease recognition site(s). Such chemically modified nucleotide(s) are compatible with incorporation by the polymerase. A suitable example of this is replacing the deoxycytidine triphosphate in the reaction buffer with C5 methyl-deoxycytidine triphosphate. For example, while Bsal is blocked by dcm methylation, which is methylation of the C5 position of the sequence 5′ CCAGG 3′ (SEQ ID NO:24), such dcm methylated sequences can still be joined by T4 ligase.

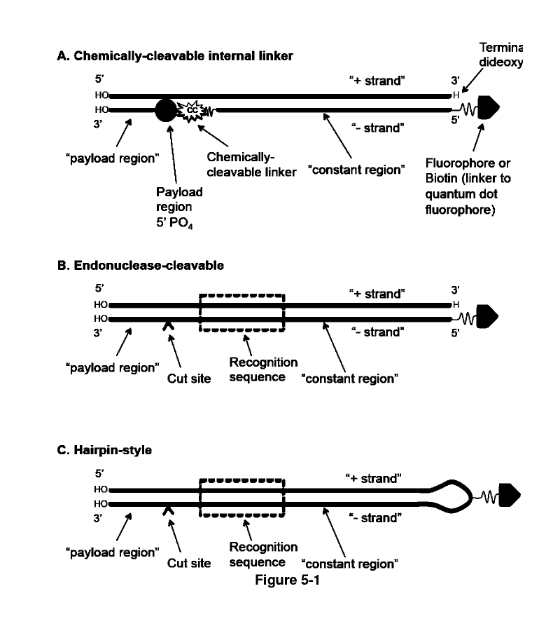

Alternate extension unit designs with terminal cleavable caps. While internal cleavable linkers are useful for extension unit designs that deliver small (i.e. 2-8 nt) payloads, optional designs with terminal caps may advantageous for the delivery of longer payload sequences (FIG. 6). In designs with terminal caps, the payload region includes the entire extension unit, and correctly oriented single addition is ensured by protection of the 5′ end of the − strand with a cleavable linker to a fluorophore, such as PC-biotin (FIG. 6A). Alternately, an appropriately designed cleavable linker added to the 3′ OH of the + strand (FIG. 6B) could also be used to ensure directional ligation of the extension unit and to track the progress of single-molecule ligation and deprotection; this scheme still requires that the − strand is phosphorylated on its 5′ end to enable subsequent ligations.

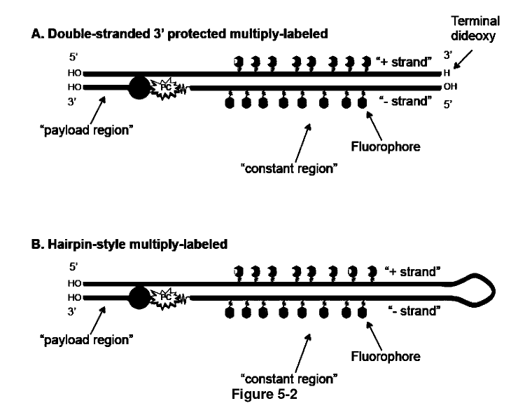

Alternate extension unit design and reaction cycle with terminal non-cleavable caps. Small changes to the reaction cycle enable design of extension units for the delivery of longer payload sequences without the need for cleavable caps: in this scheme, the extension unit − strand consists of a 5′ phosphorylated oligonucleotide containing exactly the payload sequence, and the + strand is an oligonucleotide complementary to the − strand (and possibly with a short 3′ overhang) but with its 3′ OH conjugated to a fluorophore (or biotin for binding a quantum dot, etc.) as depicted in FIG. 6C. As before, the reaction cycle begins with loading a single starter solid support, such as a magnetic bead, with a single starter DNA molecule into the reaction chamber (FIG. 7A) and proceeds as follows: (1) An excess of (fluorophore-conjugated) extension unit stock is added to the reaction chamber with T4 ligase and reaction buffer containing ATP (FIG. 7B). The ligase joins a single extension unit to the growing DNA molecule on solid support, leaving a nick at the ligation junction (FIG. 7C). (2) After an incubation period, the reaction chamber is washed with Wash Buffer to remove all extension units and enzymes (FIG. 7D). (3) Fluorescence of the reaction chamber is measured. If the fluorescence of a single extension unit is detected, it is concluded that the ligation has succeeded in joining the extension unit to the growing DNA molecule on solid support, and the reaction cycle can proceed. Otherwise, it is concluded that the ligation has failed, and is reattempted (i.e. go to step 1). (4) A strongly strand-displacing polymerase (such as phi29 polymerase) is added to the reaction chamber. Since the + strand contains a nick at the ligation junction, the polymerase will begin elongating the + strand and thereby displace the extension unit's + strand, using the extension unit's − strand as a template. Ultimately, this causes dissociation of the (fluorescently labeled) extension unit's + strand from the growing DNA molecule, and elongation of its + strand to be blunt with the newly added payload of the − strand (FIG. 7E). (5) The reaction chamber is washed with Wash Buffer to remove the dissociated extension unit + strand and enzymes (FIG. 7F). (6) Fluorescence of the reaction chamber is again measured. If the fluorescence of a single extension unit is detected, it is concluded that displacement of the extension unit's + strand has failed, and is reattempted (i.e. go to step 4). Otherwise, it is concluded that the displacement/blunting has succeeded, and the reaction cycle can proceed through another addition (i.e. go to step 1).

If the strand-displacing polymerase (step 4) does not produce blunt DNA with near perfect yield, a polymerase with no strand-displacement activity but with 3′→5′ exonuclease activity (such as T4 polymerase) may be added to the ligation reaction mix (step 1) together with dNTPs to ensure that blunting-gated ligation can proceed.

This approach and previously discussed extension unit designs for longer payloads require that custom chemically modified oligonucleotides be synthesized for each extension unit. In this approach, the − strand oligonucleotides must be phosphorylated on their 5′ end and the + strand oligonucleotides must be labeled on their 3′ end. As such, this approach is probably less cost-effective for constructing one-off sequences, and more cost-effective in a setting where many users share a large library of common “parts” that they would like to assemble in various ways. Alternately, this approach may be more cost effective if it is possible to design a scheme for chemically modifying cheap column-synthesized oligonucleotides during synthesis or at the bench. For example, it might be possible to efficiently tail the + strand oligonucleotide with a fluorescent terminator nucleotide using a polymerase followed by 3′→5′ exonuclease treatment to removed unreacted oligonucleotides, and separately phosphorylate the 5′ − strand oligonucleotide followed by some purification step. Finally, this scheme may also prove cost-effective for generating very long extension units by PCR reactions with chemically modified primers.

Recursive assembly of long DNA molecules. Recursive assembly in principle allows for assembly of DNA molecules in time logarithmic in the number of extensions instead of linear—potentially offering huge speedups for longer molecules. This speedup is achieved by recursively partitioning the target sequence into short pieces that are constructed in parallel reaction chambers; these intermediate products are used as growing DNA molecules and extension units for subsequent extension reactions, joining neighboring segments until the full-length molecule is constructed. Note that the state of the growing DNA molecule on solid support immediately before the deprotection step in the reaction cycle, if cleaved from the solid support, is functionally equivalent to an extension unit, albeit with a longer payload region—it is this property that enables recursive construction. These intermediate products could be cleaved from the beads using a type-IIs restriction enzyme that cuts at the beginning of the constructed sequence (i.e. immediately after the universal primer binding site) leaving either a blunt end (e.g. MlyI) or leaving a 3′ recessed end on the − strand, which will be repaired by the blunting polymerase in the subsequent ligation reaction. However, this scheme differs from the standard sequential addition scheme described above in that only a single extension unit molecule is available for the addition reaction, as opposed to an excess of molecules. One important implication of this difference is that the washing step following ligation must take care to store the used buffer because if the ligation did not occur during the incubation period, the single extension unit can be moved back to the reaction chamber for a second attempt. The ligation rate of a single extension unit molecule to the growing DNA on solid support will certainly be slower than the ligation rate in the presence of an excess of extension units. If this slowdown is so extreme that wipes out the benefits of the exponential reduction in the number of extension cycles, it may be necessary to amplify the intermediate to create an excess of extension units for the next ligation. The amplification reaction must create fluorescently labeled extension units suitable for scarless ligation to a growing chain; it may be necessary at this point in the assembly to switch over to extension units with caps that are cleaved by restriction enzymes, for example, adding the restriction sites into the synthesis during the conventional sequential assembly.

Extension unit stock purity. The accuracy of the final assembly product depends directly on the purity of the extension units from which it is constructed. PAGE purification of column-synthesized oligonucleotides provides sufficiently pure stocks. Similarly, all extension units must be conjugated to fluorophores; for designs that employ organic fluorophores, full-length fluorescently labeled extension unit oligonucleotides can be isolated using PAGE purification. However, for extension unit designs with oligonucleotides conjugated to quantum dots, ensuring that all extension units are bound to a (separately manufactured) quantum dot may require incubation of the oligonucleotides with an excess of quantum dots, possibly followed by some form of chromatographic purification.

Photodamage of DNA and fluorophores. Excitation light used to detect fluorescent emission from the quantum dot or small-molecule fluorophores conjugated to extension units may, in rare cases, directly damage DNA. With that in mind, fluorophores are selected that can be excited using visible wavelengths that are unlikely to damage DNA. Cleavage of a 2- nitrobenzyl photocleavable linker requires long-wave UV light, to which DNA is optically transparent. However, any irradiation might still induce DNA damage indirectly via photochemistry of the absorbing group. This can be minimized by limiting the total dose of radiation delivered to the reaction chamber and by adding free radical scavenger reagents to the buffer, and degassing the reagents to remove molecular oxygen, and/or only imaging in a buffer containing an oxygen scavenging system such as the protocatechuic acid/protocatechuate-3,4-dioxygenase. However, if it turns out that the dose of radiation necessary for efficient photolysis of the linker inevitably damages the DNA, an alternate cleavable linker must be substituted—either a chemically cleavable linker, or perhaps a photocleavable linker with absorbance at longer wavelengths.

Single-molecule detection. The DNA construction scheme described above requires accurate detection of the presence or absence of a single molecule. The “standard design” extension units are conjugated to a quantum dot or organic fluorophore and ligation and deprotection are monitored via detection of fluorescent emission. Many laboratories around the world routinely perform experiments that require detection of single-molecule fluorescence, typically using confocal or total internal reflection (TIRF) microscopy with laser illumination and electron-multiplying CCD or cooled CMOS sensors. Detection of single fluorophores requires very sensitive detectors, bright illumination, and high-quality optics. There are commercially available instruments (e.g. Pacific Biosciences SMRT sequencing machines) that can implement single-molecule detection for the practice of the invention. As such, an important challenge in building an integrated device for implementing the DNA construction scheme described will be engineering a sensitive single-molecule detector into the chip. This may require precision optics, but hopefully implementation will be simplified by the fact that the scheme described above does not require any spatial information from reaction chamber, only a binary “present/absent” answer; hopefully a single avalanche photodiode will suffice. Alternate single molecule detection schemes, such as those depending on the detection of changes in the electrical properties of the reaction (e.g. due to the presence of a single nanoparticle conjugated to the extension unit) may prove more cost effective and easier to implement than single molecule fluorescence detection.

Reagent carryover. A common challenge facing many microfluidic devices is cross-contamination via carryover of reagents when any region of the device is reused for multiple steps. In the scheme described above, carryover of extension units could reduce accuracy by causing the reaction to contain a pool of extension units with heterogeneous payloads. Carryover of small quantities of enzymes is perhaps inevitable in any microfluidic system. The DNA construction scheme described herein is completely robust to carryover of ligase and/or polymerase; ligase activity in the reaction chamber at all steps in the cycle will not lead to any side products, and blunting polymerase activity may lead to unnecessary recessing of the 3′ end of the + strand of the growing DNA molecule but this will be repaired during the next simultaneous blunting and ligation step.

Microfluidics complexity. A complete microfluidic implementation of the reaction cycle described above will have to efficiently dispense picoliter-sized droplets containing extension units from 4^{k} reservoirs and transport them to the reaction chamber. For a design with payloads of k=5, the chip will require 4⁵=1024 extension unit stocks and reservoirs, and perhaps an order of magnitude more electrodes on the chip to facilitate droplet transport. Existing microfabrication technologies routinely used to produce printed circuit boards far exceeding this level of complexity, but to my knowledge no digital microfluidic chips with a similar level of complexity have been demonstrated. A related challenge is manufacturing a chip or cartridge with sub-μL quantities of ˜1024 distinct reagent loaded onto specific locations. Changing to payloads of k=3 would reduce the library of extension units to 4³=64, reducing the required complexity of the chip to a level similar to other digital microfluidics devices reported in the literature, though at a cost of fewer nt added per cycle and potentially slower ligation kinetics. Using endonuclease-cleavable extension units, the method can further result in one nt added per cycle.

The invention having been described, the following examples are offered to illustrate the subject invention by way of illustration, not by way of limitation.

### EXAMPLE 1

### DNA Construction by Single-molecule Assembly of Capped DNA Units Bulk Experiments with Magnetic Beads

Ligation efficiency of various extension unit designs. To determine the ability of T4 Ligase to join DNA containing various photocleavable linkers near the ligation junction, the following experiments are performed:

Starter DNA is prepared from oligonucleotides are synthesized by Integrated DNA Technologies, Inc. (Coralville, Iowa):

+ strand:
(SEQ ID NO: 1)
5′{B}TGGTGTGGATCCATATAGATATCGTATCAGCTGCTGGACATTTAG
GACCGTACCG 3′
- strand:
(SEQ ID NO: 2)
5′{PO₄}CGGTACGGTCCTAAATGICCAGCAGCTGATACGATATCTATAT
GGATCCA{FAM} 3′

“{B}” denotes “biotinyl”, “{ddC}” denotes dideoxy C, “{FAM}” denotes 6-Carboxyfluorescein, “{HEX}” denotes hexachlorofluorescein, and {PO₄} denotes 5′ phosphorylated. “{PC-S}” and “{PC-L}” denote PC-Spacer and PC-Linker, respectively.

The − strand oligonucleotide is purified by HPLC. 100 pmol of each 100 μM oligonucleotide stock are annealed in 120 μL of 1x B&W (1 M NaCl, 5 mM Tris-HCl, pH 7.5) by heating to 65° C. and allowing to cool to room temperature on the bench.

Extension unit oligonucleotides are synthesized by Integrated DNA Technologies, Inc. (Coralville, Iowa) HPLC purified, except the three − strand oligonucleotides containing the PC-Linker group, which are purchased from TriLink BioTechnologies (San Diego, Calif.) PAGE-purified.

PC-Spacer 6mer + strand:
(SEQ ID NO: 3)
5′ GTGCCGNGGTCCTCTGACGATATGGAACT{ddC}3′
PC-Spacer 6mer - strand:
(SEQ ID NO: 4)
5′ {B}CAGTICCATATCGICAGAGGACC{PC-S}CGGCAC 3′
PC-Spacer 3mer + strand:
(SEQ ID NO: 5)
5′ GCGNGGTCCTCTGACGATATGGAACTG{ddC}3′
PC-Spacer 3mer - strand:
(SEQ ID NO: 6)
5′ {B}CAGTTCCATATCGTCAGAGGACC{PC-S}CGC 3′
PC-Linker 3mer + strand:
(same as PC-Spacer 3mer + strand)
PC-Linker 3mer - strand:
(SEQ ID NO: 7)
5′ {B+}CAGTICCATATCGICAGAGGACC/{PC-L+}/CGC 3′
PC-Linker 4mer + strand:
(SEQ ID NO: 8)
5′ GCTGNGGTCCTCTGACGATATGGAACT{ddC}3′
PC-Linker 4mer - strand:
(SEQ ID NO: 9)
5′ {B}CAGTTCCATATCGTCAGAGGACC/{PC-L}/CAGC 3′
PC-Linker 5mer + strand: 5′
(SEQ ID NO: 10)
GCCTGNGGTCCTCTGACGATATGGAACT{ddC}3′
PC-Linker 5mer - strand:
(SEQ ID NO: 11)
5′ {B}CAGTTCCATATCGTCAGAGGACC/{PC-L}/CAGGC 3′

6 μL of DynaBeads M-280 Streptavidin stock is washed in 1000 μL of 1x B&W with 1x BSA (New England Biolabs, Ipswich, Mass.) and resuspended in the 120 μL 1x B&W with annealed starter DNA and vortexed lightly for 15 mM 100 pmol of biotin (Sigma Aldrich, St. Louis) from a 10 mM solution is added and the tube is vortexed for 5 more minutes. Beads are washed with 1 mL 1x B&W and resuspended (to wash) in 120 μL 1x T4 ligase buffer (New England Biolabs) and split into 6 20 μL aliquots. PCR tubes are prepared with 2 μL of 2× Quick Ligation buffer (New England Biolabs, Ipswich, Mass.) and 1 μL of extension unit oligonucleotide (or ddH2O), heated to 55° C. in a thermocycler and cooled to room temperature in the block. Annealed extension units are combined with additional 2× Quick Ligation buffer and Quick Ligase (New England Biolabs) to 21 μL working volumes with 1 μL Quick Ligase stock/reaction. Beads are resuspended in the corresponding reaction mixtures and vortexed lightly for 5 minutes at room temperature. Subsequently, beads are washed twice with 100 μL of wash buffer (WB, 1x B&W with 10 mM EDTA and 1x BSA), resuspended in 20 μL of 0.01x B&W to mitigate carryover of salt ions, and finally resuspended in 50 μL ddH2O to denature the − strand from the + strand, liberating it from the beads. Tubes are incubated at 42° C. for 5 mM and the supernatant is collected and split into two 25 μL aliquots in PCR tubes; one set is placed on a UV transilluminator (365 nm setting) and irradiated for 5 mM, the other is not. 5 μL aliquots from each set are sent to Quintara Biosciences (Albany, Calif.) for fragment analysis on an ABI 3730x1 instrument (Applied Biosystems® by Life Technologies, Grand Island, N.Y.) with 1 μL of sample combined with 0.2 μL GS500LIZ size standards and 8.8 μL of HiDi formamide. Capillary electrophoresis data are analyzed using a custom pipeline written in R.

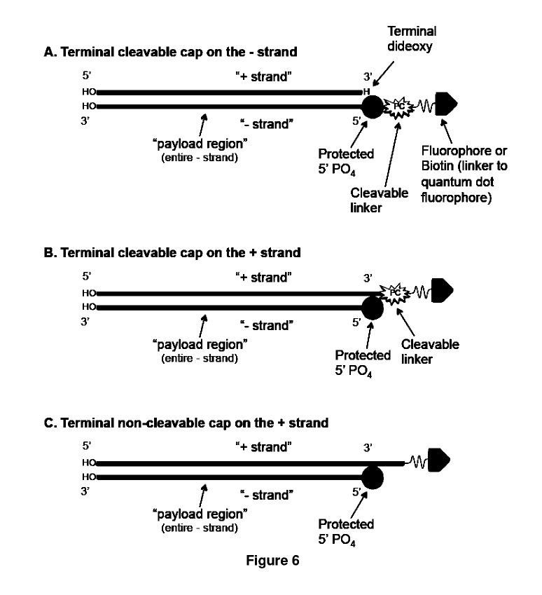

The DNA molecules ligated in this experiment are depicted in FIG. 8A. The gel-like image generated from sized, normalized capillary electrophoresis traces shown in FIG. 8B indicates successful blunt-end ligation of all extension units to the starter DNA on solid support with the exception of the PC-Spacer 3mer (bands in the “75-85” nt size range; absolute sizing is not accurate since these oligonucleotides have nonstandard chemical modifications or 5′ PO₄ groups). The PC-Linker 5mer and PC-Spacer 6mer had the highest efficiency ligation. Zooming in on the photolysis products (FIG. 8C) and taking advantage of the quantitative relative sizing offered by capillary electrophoresis, it is clear that both the PC-Spacer 6mer and PC-Linker 5mer extension units have been cleaved to produce 50+6 and 50+5 extension products; faint bands in the PC-Linker 4mer and 3mer lanes, taken together with the faint ligation product bands in the respective lanes of FIG. 8B indicate some 50+4 and 50+3 products are successfully generated as well, albeit at lower efficiency. This demonstrates successful ligation and deprotection with standard extension unit designs, albeit in the absence of quantum dots, which would be prohibitively expensive at this scale. Also notable is the presence of a significant quantity of the T4 ligase reaction intermediate 5′ adenylated DNA (AppDNA), which runs slightly “slower” than the equivalent 5′ phosphorylated DNA. This can be removed in subsequent experiments by ligation with an excess of long (>100 bp) DNA, to shift it out of the size-range of interest. These experiments can be repeated with streptavidin-bound extension units, as a proxy for the behavior of streptavidin-conjugated quantum dot-ligated extension units.

Sequential ligation of 6-mer payload extension units. To demonstrate that efficient simultaneous blunting and ligation in the same buffer is possible, and to demonstrate the complete reaction cycle, the following experiments are performed:

Starter DNA is prepared from oligonucleotides synthesized by Integrated DNA Technologies, Inc. (Coralville, Iowa).

+ strand part 1:
(SEQ ID NO: 12)
5′ {B}TGGTGTGGATCCATATAGATATCG{ddC}
+ strand part 2:
(SEQ ID NO: 13)
5′ {HEX}AAAAAAAAAATITTITTITTATCAGCTGCTGGACATTTAGG
ACC 3′
- strand: same as above

100 pmol of all three 100 μM starter DNA oligonucleotide stocks are annealed in 240 μL of 1x B&W as described above. 12 μL of DynaBeads M-280 Streptavidin stock is washed in 1000 μL of 1x B&W with 1x BSA (New England Biolabs, Ipswich, Mass.) and resuspended in the 240 μL 1x B&W with annealed starter DNA and vortexed lightly for 15 min, and blocked with free biotin as described above. Subsequently, beads are resuspended in 240 μL of WB and a 40 μL aliquot is removed and stored on ice. 80 ↑L of 1x Quick Ligation buffer with 25 μM PC-Spacer 6mer extension unit is prepared by adding 20 μL of each 100 μM each oligonucleotide stock to 40 μL of 2x Quick Ligation buffer and gently annealed by heating to 42° C. and allowing to cool to room temperature.

420 μL of Reaction buffer (RB) is prepared by combining the 80 μL of annealed PC-Spacer 6mer solution 100 μL of 2x Quick Ligation buffer, 4 μL 10 mM dNTPs (Fermentas, Waltham) (final concentration 100 μM), 96 μL ddH2O, 20 μL of Klenow (New England Biolabs) stock diluted 1:10 in Quick Ligation buffer, and 20 μL of Quick Ligase stock.

Remaining beads are resuspended in 200 μL T4 ligase buffer (to wash) and then resuspended in 200 μL of RB for a 5 minute incubation vortexing every ˜2 mM to keep the beads suspended. After 5 minutes, 200 μL of 20 mM EDTA is added to stop the reactions and beads are resuspended in 200 μL of WB. Beads are washed again in 200 μL of WB, and a 40 μL aliquot is taken and stored on ice.

Beads are resuspended in 160 μL of photolysis buffer (PB, 0.01x B&W with 10 mM EDTA and 1x BSA), transferred to a glass GCMS vial and irradiated on a UV transilluminator (365 nm setting) for 5 minutes, vortexing every minute. Beads are subsequently transferred to a fresh tube and washed twice in 160 μL of PB, and a 40 μL aliquot is removed and stored on ice.

Beads are resuspended in 120 μL T4 ligase buffer (to wash) and the process described above is repeated with the volumes scaled appropriately, taking 40 μL aliquots after ligation and photolysis steps.

Finally, all stored 40 μL aliquots are split into two 20 μL aliquots, one of which is saved for a different set of experiments. Each remaining aliquot is washed in 100 μL PB and washed again in 20 μL 0.01x B&W before resuspension in 50 μL ddH2O for elution of the DNA from the beads. Tubes are incubated at 42° C. for 5 minutes and the supernatant is collected, and 5 μL aliquots are sent to Quintara Biosciences for fragment analysis as described above. Capillary electrophoresis data are analyzed as above.

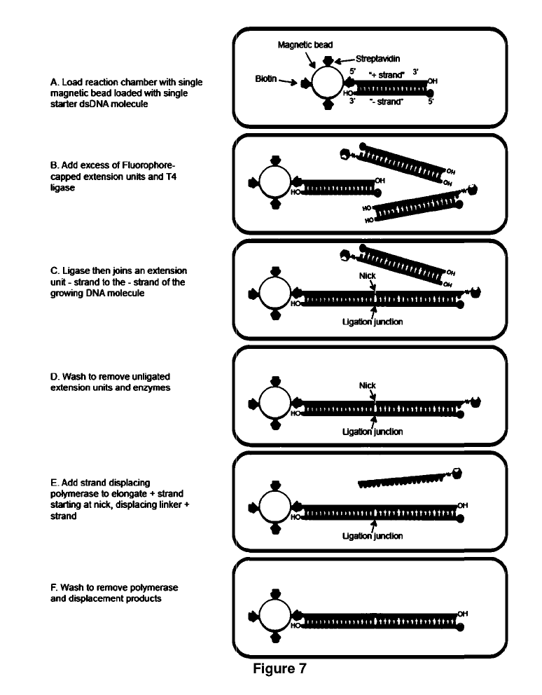

The DNA molecules joined in this experiment, a 6 nt 3′-recessed fluorescently-labeled starter DNA on solid support, and the PC-Spacer 6mer extension unit, are depicted in FIG. 9A. The gel-like image generated from sized capillary electrophoresis traces shown in FIG. 9 indicates that simultaneous elongation of the + strand (i.e. blunting) by the Klenow fragment polymerase is compatible with efficient blunt-end ligation of the elongation products by T4 ligase with the PC-spacer extension unit. Photolysis of the ligation products on the beads shows a clear +6 extension product, which serves as a substrate for subsequent simultaneous blunting and ligation with a new extension unit. A second photolysis reveals a clear, albeit faint +12 extension product; thus, these results, representing two complete cycles of the extension process, demonstrate the feasibility of the complete extension cycle. It may be worthwhile to repeat these experiments under conditions that would permit detection of third and fourth cycle extension products, to demonstrate the feasibility of additional cycles. As above, a detectible amount of the AppDNA ligation intermediate accumulates during each ligation step; this will not occur in the single-molecule implementation because ligation reaction to the single growing DNA molecule will be tracked until completion.

### Single-molecule Experiments in Rudimentary Flow Cells

Construction of multiply-labeled hairpin extension units. Multiply-labeled extension units are constructed from oligonucleotides containing the photo-cleavable linker and payload and a 199 nt hairpin containing 32 sites for labeling with Alexa 647 fluorophores. Oligonucleotides are purchased from IDT (Coralville, Iowa):

+ strand:
(SEQ ID NO: 14)
5′ GTGCCGtGGTCCTCTGACGATATGGAACTGGTCCGGGA 3′
- strand:
(SEQ ID NO: 15)
5′ {PO₄}GGACCAGTTCCATATCGTCAGAGGACC{PC-S}CGGCAC 3′
hairpin:
(SEQ ID NO: 16)
5′ {PO₄}attaatatatttattataaaGaaatGtattGaataGattaGtt
taGtataGaattGtaatGatttGatatGaataGtaaaGtttaGtttaGaaa
tGtttGatttGtaaaGtaaaGtttaGtattGatatGaaatGattaGaattG
tataGtaaaGtaatGtattGaataGatttGattaatatatttattataaa
TCCC

The hairpin is labeled at the G bases using the Alexa 647 ULYSIS labeling kit (Life Technologies, Carlsbad, Calif.) following the manufacturer's instructions and purified using a Micro Bio-Spin P30 column (Bio-Rad, Hercules, Calif.), and then dialyzed into deionized water. Degree-of-labeling is computed from absorbance at 260 nm (DNA) and 650 nm (Alexa 647) using a NanoDrop ND-1000 spectrophotometer. Labeled hairpin is ligated with a 10-fold molar excess of extension unit +and − strand oligonucleotides using the Quick Ligation kit and cleaned up on Zymo DNA Clean and Concentrator-5 columns (Zymo Research, Irvine, Calif.). Assembled extension units are analyzed by capillary electrophoresis on an ABI 3730x1 instrument.

Construction of pre-extended starter DNA. Oligonucleotides forming the 50 bp 5′ biotinylated/5′ phosphorylated starter dsDNA are diluted to 10 μM in 1x Quick Ligation buffer and 10 μL is annealed by heating to 65° C. in a thermocycler and allowing to cool to RT. Fluorescent extension units are diluted to 100 nM in 1x Quick Ligation buffer and 10 μL are added to the annealed starter dsDNA together with 1 μL of Quick Ligase and incubated for 5 minutes at RT. Ligation products are analyzed by (unstained) 3% agarose gel electrophoresis, and the highest molecular weight fluorescent band is excised and purified using a Zymoclean Gel DNA Recovery Kit (Zymo Research, Irvine, Calif.).

Imaging of discrete immobilized starter DNA molecules. Sample chambers are constructed using the method of Joo and Ha (Joo C, Ha T. Preparing sample chambers for single-molecule FRET. Cold Spring Harb Protoc. 2012 Oct. 1; 2012(10):1104-8. doi: 10.1101/pdb.prot071530) using PEG/PEG-biotin “Bio_01” functionalized coverslips (Microsurfaces, Englewood, N.J.). Flow chambers are filled with 0.2 mg/mL streptavidin (New England Biolabs) in buffer T50 (10 mM Tris-HCl, 50 mM NaCl) and incubated for 1 minute before washing with three sample volumes of T50. Pre-extended fluorescent starter DNA molecules are diluted to 100 μM in T50 and flowed into the sample chamber. At this concentration, there should be ˜6 molecules/m², or ˜3800 in a 25×25 μm viewing area, if all molecules bind to the surface. After a 5 minute incubation, the sample chamber is washed with three sample volumes of T50 and imaged through a 100x oil immersion objective on a Leica DM4000B fluorescence microscope using a mercury lamp using a “Cy5” 620/60 nm excitation 700/75 nm emission filter set. If the image shows point-like fluorescent emitters with a density of roughly 6/μm², then it is concluded that the emitters are individual pre-extended fluorescent starter DNA molecules.

Photo-deprotection in situ. A sample chamber is prepared as described above and loaded with non-photo-cleavable pre-extended fluorescent starter DNA molecules and imaged using the Cy5 filter set. The sample chamber is then irradiated with long-wave UV light using an AT350/50x “DAPI” filter set for a 5 minutes. Subsequently, the reaction chamber is washed with three sample volumes of T50, and again imaged using the Cy5 filter set. If all spots corresponding to individual immobilized non-photo-cleavable starter DNA molecules are still present, then it is concluded that the fluorophores and DNA are stable during the irradiation used for deprotection. This is expected because neither the DNA nor the fluorophores absorb 300-350 nm light. A new sample chamber is loaded with photo-clevable pre-extended fluorescent starter DNA molecules and imaged as above. Again, the sample chamber is irradiated with long-wave UV light, and washed with 3 volumes of T50. If at least 90% of spots have disappeared, then it is concluded that the irradiation protocol successfully deprotected the DNA molecules.

High-stringency washing of unreacted extension units. A sample chamber is prepared as described above but (non-fluorescent) non-phosphorylated starter DNA is substituted in place of the pre-extended starter DNA. The sample chamber is imaged to determine background fluorescence. RB containing 5 μM fluorescent extension units is flowed into the sample chamber and allowed to incubate for 5 minutes before washing with 3 sample volumes of T50. (No ligation should take place because the immobilized DNA is not 5′ phosphorylated.) The sample chamber is again imaged as described above. If the fluorescence of the sample chamber returns to baseline levels, then it is concluded that washing has successfully removed all unreacted fluorescent extension units from the functionalized surface.

Single-molecule detection of extension in situ. A sample chamber is prepared as above and loaded with photo-cleavable pre-extended fluorescent starter DNA molecules, imaged using the Cy5 filter. Sample chambers are irradiated and washed as described above to photo-deprotect the DNA molecules, leaving exposed 5′ phosphates suitable for extension. The sample chamber is filled with RB containing 5 μM fluorescent extension unit. After a set incubation time, the reaction chamber is washed with 3 sample volumes of T50 and imaged using the Cy5 filter. If a new spot appears in at least 90% of the locations where a spot disappeared following photo-deprotection, then it is concluded that the extension protocol detectably extended single DNA molecules on the surface. Subsequently, the sample chamber is again irradiated and washed to confirm that the new spots are indeed photo-cleavable extension products.

Multiple in situ extension/deprotection cycles. A sample chamber is prepared as above and loaded with photo-cleavable pre-extended fluorescent starter DNA molecules, and subjected to deprotection and extension as described above. The process is repeated 3 more times, recording images of single molecules between each step. If the deprotection and extension steps have a similar success rate in all cycles, then it is concluded that the reaction scheme is feasible, and that it is possible to track single molecules through multiple extension/deprotection cycles.

### Full System Integration Tests

Bead dispensing and single-molecule detection. 100 μg of Dynabeads M-280 Streptavidin (10 μL of stock solution, ˜6×10⁶ beads) are washed in 1 mL of 1x B&W and resuspended in 20 μL of 1x B&W with 25 fM pre-extended (fluorescent, photo-cleavable) biotinylated starter DNA, corresponding to a ˜20-fold molar excess of beads over DNA; at this dilution, if all DNA molecules bound to the beads, roughly 95.1% of beads would have zero DNA molecules bound, 4.8% would have one DNA molecule bound, and roughly 0.1% would have two or more DNA molecules bound. Beads are vortexed lightly for 20 minutes at RT for binding and then washed twice in 1 mL of WB and resuspended in 60 μL of WB, corresponding to ˜10⁵ beads/μL. An aliquot of beads is further diluted by a factor of 2×10⁸ in WB in four serial dilutions in order to prepare a suspension with ˜2000 beads/μL such that the probability that a 10 nL volume contains a bead is roughly 1/20.

The microfluidic device (FIG. 4) is mounted on a modified epifluorescence microscope and primed with silicone oil. 10 μL of diluted beads are loaded into the “Pre-loaded bead” input port of the microfluidic device and are subjected to continuous mixing by transport between two adjacent pads. 10 nL droplets are dispensed from the reservoir and transported to the reaction chamber. Roughly 95% of dispensed droplets should contain zero beads, 4.8% should contain a single bead, and 0.1% should contain two beads. Of the droplets containing beads, roughly 95% should contain only a bead and no DNA, 4.8% should contain a single DNA molecule (0.23% of all droplets, or ˜1 in 430), and 0.1% should contain a bead with multiple DNA molecules. The device is monitored continuously to ensure successful transport of droplets. After each step, the reaction chamber is imaged in brightfield and fluorescence mode using a 650 nm laser diode and a 705/72 nm emission filter. An algorithm is applied to compute a summary statistic of the fluorescence indicating the presence or absence of a single fluorescent DNA molecule on the bead (e.g. total fluorescence is greater than some threshold, or there exists a spot with a minimum size with peak fluorescence greater than some threshold, etc.) and frames are examined manually to ensure that the algorithm is correctly classifying the presence or absence of a single DNA molecule on the bead in the reaction chamber. If the algorithm's error rate is less than some acceptable value (e.g. 1 in 10⁵), then it is established that the system can dispense single beads and detect single fluorescent DNA molecules on the beads.

In some embodiments, the microfluidic device for single-molecule DNA construction comprises one or more elements as shown in FIG. 4B. The device comprises of micro-channels and computer-controlled micro-valves made of PDMS bonded to a coverslip. The device comprises input ports for all reagents required for single-molecule DNA construction, including, but not limited to, the buffers, ExUs, and starter DNA. A designated channel serves as a nL-scale reaction chamber where single-molecule DNA construction takes place. Optionally, the device is mounted on an automated pseudo-TIRF microscope for imaging single-Cy3 fluorescence on an EMCCD detector. Optionally, an algorithm dispenses reagents to the reaction chamber to direct construction of the specified DNA sequence, monitoring progress using the detector and repeating failed steps as necessary.

In a different implementation of the device, the microscope is replaced by an integrated system containing custom fluorescence excitation and detection photonics (such as microlenses, an LED, and an avalanche photodiode). In that implementation, an analogous algorithm is required for classification of the presence or absence of fluorescent DNA in the reaction chamber based on the signal from the detector.

Bead trapping and high-stringency washing of soluble extension units. It is crucial that washing the reaction chamber removes all soluble fluorescent extension units (i.e. all except for one that has successfully ligated to the growing DNA molecule on the bead.) To test this, a bead loaded with a single non-fluorescent starter DNA molecule without a 5′ phosphate is transported to the reaction chamber and pinned to the chamber by activation of an integrated electromagnet. The presence of the bead is confirmed using brightfield microscopy, and 10 nL droplets of WB are transported to the reaction chamber as the liquid in the reaction chamber is transported to the waste port with the electromagnet active. If the bead remains in the reaction chamber during washing, then it is established that the system can trap a single bead and wash the reaction chamber. Subsequently, RB containing 5 μM fluorescent extension unit is loaded into the “Enzymes/MM input port”, and 10 nL droplets are dispensed to replace the contents of the reaction chamber while continuing to trap the bead. Since the DNA molecule on the bead is not phosphorylated, it cannot be covalently joined to an extension unit. After 5 minutes of incubation, the reaction chamber is washed three times with 10 nL droplets of WB, and the cycle is repeated to gather statistics. If baseline fluorescence is detected following washing (i.e. the algorithm indicates that no DNA molecules are present) at some acceptably high rate (e.g. 0.99999), then it is established that the system can wash the reaction chamber of all soluble fluorescent extension units with high stringency.

Photo-deprotection in situ. Droplets containing beads pre-loaded with non-photo-cleavable pre-extended fluorescent starter DNA molecules are transported to the reaction chamber until a bead with a single fluorescent DNA molecule is captured, as described above. The reaction chamber is then irradiated with long-wave UV light (e.g. using a mercury lamp through a AT350/50x DAPI filter) for a set exposure. Subsequently, the reaction chamber is washed with WB as described above, and the fluorescence of the reaction chamber is measured. Finally, the bead is transported to the waste port, and the cycle is repeated to gather statistics. If fluorescence is detected following washing at some acceptably high rate (e.g. 0.99999), then it is established that the fluorophore and DNA are stable during deprotection. This is expected because neither the DNA nor the fluorophore absorb 300-350 nm light.

Next, the reaction chamber is loaded with a bead containing a photo-cleavable pre-extended fluorescent DNA molecule, and the reaction chamber is irradiated and washed as described above. The bead is transported to the waste port, and cycle is repeated to gather statistics. If no fluorescence is detected following washing at some acceptably high rate (e.g. 0.6), then it is established that the irradiation protocol successfully deprotected the single DNA molecule on the bead.

In an system with integrated custom optics, the photolysis irradiation could be generated by a 330 nm LED focused onto the reaction chamber.

Single-molecule detection of extension in situ. A single bead containing a single photo-cleavable fluorescent pre-extended starter DNA molecule is loaded into the reaction chamber and photo-deprotected as described above, leaving a single bead with a 5′ phosphorylated DNA molecule suitable for extension. With the bead trapped, the reaction chamber liquid is replaced with RB containing 5 μM fluorescent extension unit. After a set incubation time, the reaction chamber is washed with WB as described above, and the fluorescence is recorded. If fluorescence is detected, then the reaction chamber is irradiated and washed as described above, and the fluorescence is recorded. Finally, the bead is transported to the waste port, and the cycle is repeated to gather statistics. If fluorescence is detected following washing at some acceptably high rate (e.g. 0.6), and subsequently no fluorescence is detected after photo-deprotection at some acceptably high rate (e.g. 0.6), then it is established that the system can detect the extension of the single DNA molecule on the bead.

Automation of multiple extension/deprotection cycles in situ. A single bead containing a single photo-cleavable fluorescent pre-extended starter DNA molecule is loaded into the reaction chamber and photo-deprotected as described above. The “Enzymes/MM” input port is loaded with 1.25x RB (without extension units), and a chamber of the chip is loaded with 50 μLIM fluorescent extension unit in deionized water. A control algorithm implementing the scheme depicted in FIG. 1 dispenses droplets totaling 1 nL of extension unit and 9 nL of 1.25x RB, merges the droplets and mixes by transporting back and forth, and then replaces the reaction chamber with the 1x RB mixture. After a set incubation time, the reaction chamber is washed as described above, and fluorescence is measured to determine if the extension is successful. If extension failed, the control algorithm repeats the extension step. Otherwise, the control algorithm proceeds through the photo-deprotection and wash steps. If fluorescence is still detected, photo-deprotection and washing are reattempted. Otherwise, the control algorithm proceeds with another extension step. This process is repeated until 100 extension/deprotection cycles have completed or until some time limit is reached. Finally, the bead is transported to the waste port, and the process is repeated to gather statistics. If the control algorithm successfully completes 100 extension/deprotection cycles in the time allotted at some acceptably high rate (e.g. 0.9), then it is established that the system can autonomously control the extension/deprotection cycle with high fidelity.

Next, 8 chambers on the device are loaded with 50 μM stocks of fluorescent extension units in deionized water with 8 distinct payload regions. The control algorithm chooses a random sequence of 100 extensions using the 8 different extension units, loads a starter bead into the reaction chamber, and proceeds as above. Finally, the bead is transported to the waste port, and the process is repeated to gather statistics. If the control algorithm successfully completes 100 extension/deprotection cycles in the time allotted at some acceptably high rate (e.g. 0.9) regardless of the sequence of extension units used, then it is established that the system can autonomously control the extension/deprotection cycle with high fidelity using several distinct extension units.

Single-molecule amplification and sequence verification. A sequence of 100 extensions using 8 extension units is chosen, and the control algorithm is set to automate the extension/deprotection cycle. If it completes, the electromagnet is deactivated and bead is transported to the output port. 10 μL of deionized water is added to the output port reservoir and the contents are transferred to a PCR tube containing 25 picomoles of the forward and reverse universal primers each. A standard 50 μL PCR reaction is set up using Phusion polymerase (New England Biolabs, Ipswich, Mass.) following the manufacturer's instructions but with 40 cycles. The PCR product is cleaned up on a Zymo DNA Clean and Concentrator-5 column and analyzed by 1% agarose gel electrophoresis and Sanger Sequencing on an ABI 3730x1 DNA analysis system (Applied Biosystems, Foster City, Calif.). If the sequence of the amplified DNA corresponds to the sequence of the 100 payloads specified to the control algorithm, then it is concluded that the system can autonomously synthesize a single DNA molecule with a specified sequence. This implies that, equipped with the full complement of extension units covering all possible payload sequences, the system could synthesize DNA with any specified sequence.

If necessary, an on-chip isothermal amplification step, (e.g. nick-mediated isothermal amplification,) is performed in the reaction chamber before transfer of the synthesized DNA to the output port.

### EXAMPLE 2

### DNA Construction by Single-molecule Assembly

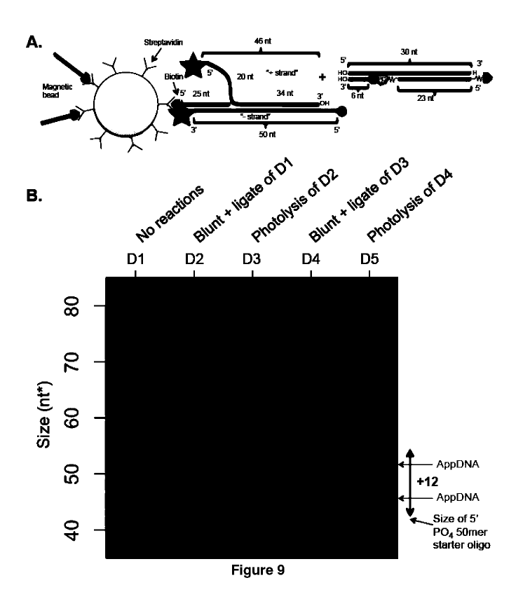

1. ExU designs, including modified bases as cleavable linkers. Hairpin ExUs with modified nucleobases that serve as cleavable linkers are synthesized and assayed. In the examples below, a single deoxyuridine base is incorporated into the oligo during synthesis serves as a cleavable linker, where the cleaving reagent is actually a combination of enzymes known as the “USER Enzyme”—E. coli uracil DNA glycosylase (UDG), which cleaves the glycosidic bond between the uracil base and the deoxyribose, and e. coli endonuclease VIII (EndoVIII), which cleaves the DNA backbone at the generated abasic site, leaving 5′ PO₄ and 3′ PO₄ termini. The USER Enzyme is chosen because it is commercially available (from NEB). Equivalent deprotection cocktails could have also created by combining UDG with another enzyme that cleaves abasic sites in DNA leaving a 5′ PO₄ terminus, including E. coli Endonuclease III (EndoIII) or Fpg (formamidopyrimidine DNA glycosylase), or the human enzymes OGG1 or NEIL. Different modified nucleobases than uracil as a cleavable linker could have also used, combined with any enzyme that cleaves the backbone at the site of the modified nucleobase leaving a 5′ PO₄ terminus. For example, the endonucleases mentioned above have intrinsic N-glycosylase activity of varying degrees towards oxidized nucleobases including: 8-oxo-7,8-dihydroguanine, 5,6-dihydrouracil, thymine glycol, 4,6-diamino-5-formamidopyrimidine, 2,6-diamino-4-hydroxy-5-formamidopyrimidine, 7,8-dihyro-8-oxoguanine, 5,6-dihydroxycytosine, 5,6-dihydroxyuracil, cytosine glycol, uracil glycol, 5-hydroxycytosine, 5-hydroxyuracil, 5,6-dihydrouracil, 5-hydroxy-6-hydrouracil, 5,6-dihydrothymine, and 5-hydroxy-6-hydrothymine. Such an endonuclease could cleave an abovementioned modified nucleobase via its N-glycosylase activity and immediately cleave the backbone of the DNA via its AP-lyase activity, eliminating the need for a separate enzyme such as UDG for N-glycosylase activity. See FIG. 11.

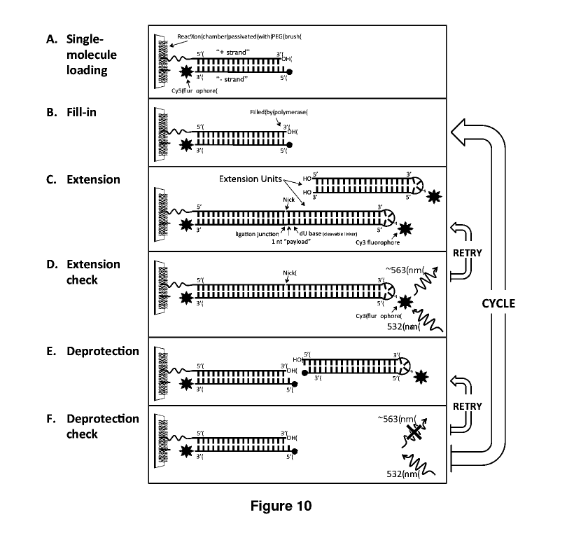

2. Two complete reaction cycles demonstration. Bulk experiments in solution are performed to demonstrate that the reaction cycle can be iterated to extend DNA by a defined number of bases per cycle. In these experiments, a 56 bp (blunt) phosphorylated dsDNA molecule labeled with a 5′ tetrochlorofluorescein (TET) on its + strand and a 3′ fluorescein (FAM) on its − strand is ligated with the iCy3 GT ExU described above. Starter + strand sequence: 5′ {TET}-aacaaggtaccaacacaccacccacccaacccataatcaatctttcacctccaccc 3′ (SEQ ID NO:26); starter − strand sequence: 5′ PO₄-gggtggaggtgaaagattgattatgggttgggtgggtggtgtgttggtaccttgtt-{FAM} 3′ (SEQ ID NO:27). All oligos are synthesized by Integrated DNA Technologies (Coralville, Iowa). The starter is annealed by heating to 55° C. and cooling to 25° C. linearly for 20 min at 1 μM in the Elution Buffer (EB) of the Zymo DNA Clean and Concentrator Kit (Zymo Research). For the first step of the first iteration of the reaction cycle, a 20 μL Quick Ligation reaction (NEB) is prepared with 50 nM annealed starter and 1 μM iCy3 GT ExU according to the manufacturer's protocol. After a 1 minute incubation at room temperature (RT), the reaction is quenched with Binding Buffer (BB) of the Zymo Kit and immediately purified, eluting with 12 μL of EB. (Note that the Zymo column has a strong length-dependence of retention below 50 bp; the 56 bp starter is retained ˜75% whereas the 17 bp (hairpin) ExU is retained <%0.1, resulting in ˜1 nM ExU carryover into the subsequent reaction.) 1 μL of purified products is saved, and 10 μL of products are added to a 20 μL deprotection reaction in 1x CutSmart buffer with 1 μL of USER Enzyme (NEB), which is incubated for 5 minutes at 37° C. Without further purification, the reaction is cooled to RT and combined with an equal volume of 1x CutSmart buffer containing 66 μM dNTPs and 1 unit of Klenow polymerase (NEB), resulting in a 40 μL fill-in reaction containing 33 μM dNTPs. The fill-in reaction is incubated for 1 minute at RT and then purified using the Zymo kit and eluting with 10 μL of EB, thus completing the first cycle, and 1 μL of purified DNA is saved. For the second ligation, 8 μL of the previous step's products are ligated with 1 μM iCy3 GT ExU for 1 minute in a 20 μL Quick Ligation reaction as described herein and purified using the Zymo kit, eluting with 12 μL of EB. Subsequently, 10 μL of these ligation products are subjected to deprotection and fill-in as described herein, and purified using the Zymo kit, eluting with 10 μL of EB. Saved aliquots of purified DNA are diluted to ˜5 nM with EB, assuming 75% yield from the PCR cleanups, and are further diluted 5-fold with a 1 nM solution of a 27 nt 5′ FAM-labeled oligo that served as a loading control for capillary electrophoresis (C.E.). The diluted samples are submitted to the UC Berkeley DNA Sequencing Facility for denaturing C.E., where 5 μL of each is combined with 14.5 μL of HiDi Formamide and 0.5 μL of GeneScan 600 LIZ size standard (Life Technologies), and are analyzed on an ABI 3730x1 (Applied Biosystems) using the DS-33 dye set. An additional calibration sample is submitted with 1 nM of the three oligos, and the spectral mixing matrix is estimated from 2-nt windows surround the peaks using a custom R script. C.E. data are then spectrally unmixed using the estimated matrix, and chromatograms are resealed according the LIZ size standard peaks using a custom R workflow. See FIG. 12.

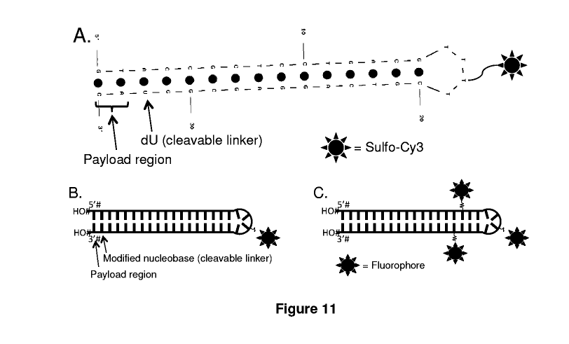

3. Discrimination between ligated ExUs and nonspecifically-bound ExUs by fluorescence fluctuations. In the present system, single-molecule fluorescence detection of the labeled ExUs is used to infer successful or failed ligation and deprotection by looking for the appearance and disappearance of fluorescence at the site where the starter DNA molecule is anchored. A naive algorithm for determining the success of the ligation step simply takes an image of the reaction chamber from a single exposure and calls a ligation successful if there is a fluorescent spot in a particular region of the image. As such, if an ExU were to bind nonspecifically to the surface of the reaction chamber very close to the spot where the starter DNA molecule is anchored, the naive algorithm would mistake it for a successful ligation. However, it is expected that an ExU nonspecifically bound to the surface of the reaction chamber would have different spatial dynamics than an ExU ligated to the end of the growing starter DNA molecule—for example, an ExU bound to the surface might have smaller spatial fluctuations than an ExU ligated to the growing DNA molecule, whose flexibility would permit its conjugated fluorophore(s) to diffuse around a larger volume. This “tethered” diffusion may be detected by examining the fluctuations of the center of the spot in the fluorescence image, and/or by examining fluctuations in the spot intensity. If correctly ligated ExUs indeed have distinctive fluorescence dynamics compared to non-specifically bound ExUs, it would enable a more sophisticated decision algorithm for deciding whether a ligation step was successful. Furthermore, this improved ability to distinguish ligated ExUs from nonspecifically bound ExUs lowers the passivation requirements of the reaction chamber, and enables the system to make accurate decisions in the presence of greater nonspecific ExU binding. See FIG. 13.

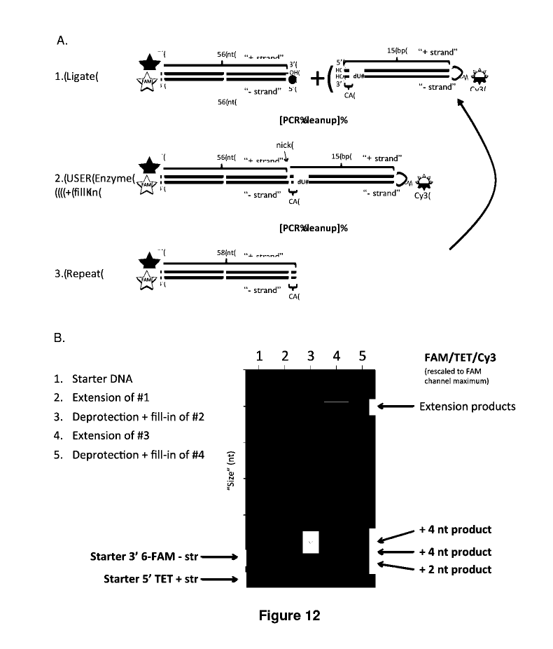

4. Hybridization tag single-molecule loading strategy. The strategy comprises the following: (1) Incubate with starter concentration/duration s.t. E[bound molecule]=1. (2) Map single-molecule fluorescence in entire reaction chamber. (3) If no molecule found, repeat incubation. (4) If 2 or more molecules found, denature hyb. Tag and flush, and repeat incubation. (5) If exactly one molecule found, proceed to DNA construction. (Nick is sealed during the first ligation.) FIG. 14 shows a single-molecule loading strategy. Single-molecule DNA construction requires that the reaction chamber of our device be loaded with a starter DNA molecule on solid support. In this particular scheme for single-molecule DNA construction, the reaction chamber is loaded with exactly one starter DNA molecule. This is achieved using a strategy similar to how one determines if a ligation step of our reaction cycle is successful: a single starter DNA molecule, which is ultimately to be extended into the full-length synthon, comprises a fluorescently-labeled double-stranded DNA molecule with a ˜25 nt 3′ overhang on one end that serves as a hybridization tag. The opposite end of the molecule is blunt and 5′ phosphorylated, or has been pre-ligated to an ExU. The reaction chamber, which is passivated with a high-density polyethylene-glycol (PEG) brush, has a low density ˜25 nt oligos that are covalently anchored via their 5′ termini to PEG molecules. These anchored oligos are complementary to the 25 nt overhang of the starter, and thus incubation of a dilute solution of starter in the reaction chamber results in some starter molecules hybridizing to the anchored oligos and thus becoming immobilized on the surface of the reaction chamber. After thoroughly flushing the reaction chamber, the number of captured starter molecules immobilized is approximately Poisson-distributed. A fixed duration incubation with the optimal concentration of starter for single-molecule loading results in one molecule binding with probability ˜0.37, no molecules binding also with probability ˜0.37, and two or more molecules binding with probability ˜0.26. The number of starter molecules bound is determined by single-molecule fluorescence imaging of a fluorophore conjugated to the starter. (If the starter is pre-ligated to an ExU, one can take advantage of the robust single-molecule ExU detection system described above.) If no starter molecules are identified, the incubation is repeated. If multiple molecules are identified, the hybridization tag is denatured (e.g. by introduction of formamide or urea), the reaction chamber is thoroughly flushed, and the incubation is repeated. If exactly one molecule is identified, the scheme for single-molecule construction may begin. Note that during the first ligation step of construction, the nick on the + strand will be sealed, covalently anchoring the starter DNA molecule to the surface. This nick can be regenerated (for reusing the device) by use of a sequence-specific nicking endonuclease, such as Nt.BbvCI.

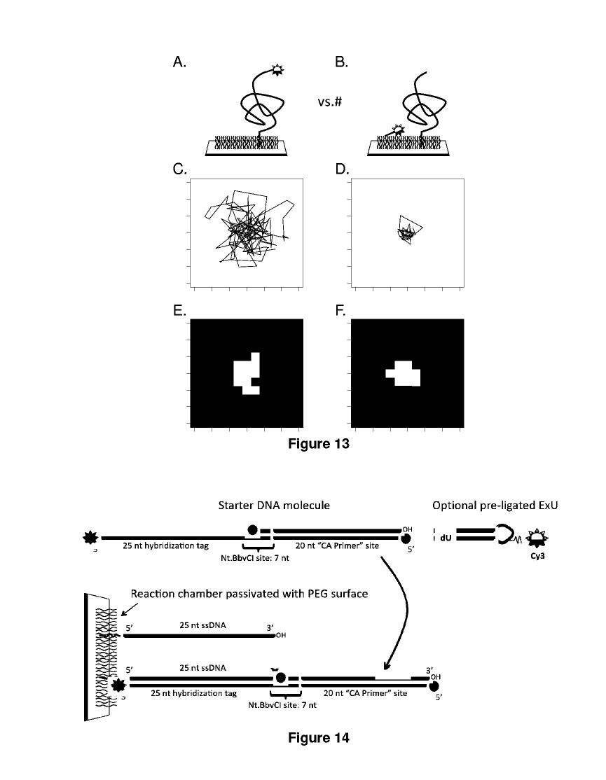

5. Two-stage single-molecule amplification strategy. FIG. 15 shows a post-synthesis single-molecule isothermal linear amplification scheme. After the starter DNA molecule has been extended to the full-length synthon, it must be liberated from the surface and amplified to obtain a useful quantity of DNA in solution. It is expected that it would be very difficult to reliably detach the precious single molecule from the surface and transport it off the chip into a conventional PCR reaction, so a two-phase amplification protocol is developed in which the single molecule is first amplified in situ, generating a sufficient quantity of DNA for transport off the chip. In the first phase, the immobilized single molecule is amplified linearly under isothermal conditions by the combined action of a sequence-specific nicking endonuclease, such as Nt.BbvCI, and a high-fidelity polymerase with high strand-displacement activity, such as phi29 polymerase. Nt.BbvCI nicks the single DNA molecule at the part of the sequence corresponding to the end of the hybridization tag used initially to immobilize the “starter DNA molecule” (see FIG. 14), effectively creating a primer-template junction for the polymerase to initiate elongation. As the polymerase proceeds with elongation, it displaces the + strand of the template into solution. Meanwhile, the nicking endonuclease can regenerate the nick at the 5′ end of the template, creating another primer-template junction from which the polymerase can initiate. This process repeats continuously through the incubation period, generating soluble ssDNA of the + strand only, which can subsequently be transported off the chip for standard thermocycling PCR amplification with a standardized primer such as the “CA primer” (Yehezkel et al. Methods Mol Biol. 2012;852:35-47) depicted above, whose binding site is present at the 5′ end of the ssDNA and whose reverse-complement is present at the 3′ end.

6. “Multiple single molecules”+post-selection of correct synthons. The reaction chamber as loaded with a single starter DNA molecule. The present reaction cycle iteratively extends the single starter DNA molecule until the desired sequence is constructed, ensuring success at each step by monitoring single-molecule fluorescence and reattempting failed steps. Finally, the single constructed molecule is be amplified to yield a useful quantity of DNA. Since there is only a single molecule serving as a template for amplification, the amplified products should be homogenous, (up to the accuracy of the high-fidelity polymerase(s) used.)

An alternative “single molecule DNA construction” scheme is possible using the same reaction cycle, but instead of loading the reaction chamber with a single starter DNA molecule, it is loaded with hundreds of spatially resolvable but otherwise identical starter DNA molecules. The reaction cycle then proceeds previously described, recording single-molecule fluorescence images of the reaction chamber at each step to determine which of the growing molecules have successfully been extended or deprotected. However, in this scheme, there is no concept of reattempting “failed” steps, because in each step some of the molecules will succeed and some will fail. After the reaction cycle has been iterated with all of the payloads required to construct the desired sequence, the series of recorded of images is examined to identify which of the growing molecules have completed every single extension and deprotection step, and thus are expected to contain the full-length desired sequence. If no such molecules are identified, then the synthesis has failed. However, if even one “correct” molecule is identified, it can be selectively amplified in situ for recovery from the device.

There are several approaches for how one can selectively amplify the “correct” molecules. These approaches described below use tightly focused light to selectively release, activate, or destroy individual molecules to ensure that only the correct molecules are amplified.

(1) In the first approach, the correct molecules are selectively released from the surface via cleavage of a photocleaveable linker through which they are anchored to the solid support. For example, if the starter DNA were immobilized via a PC-biotin modification, it could be liberated into solution via irradiation with focused UV light. The solution in the reaction chamber could then be transferred to another chamber where the amplification reagents could be introduced. This approach risks loss of the soluble DNA molecules to nonspecific binding during the transfer of the reaction chamber contents.

(2) In the second approach, a photocleavable moiety is employed on the starter DNA that directly or indirectly prevents amplification. One example of a photocleavable moiety that directly prevents amplification is an incorporated photo-reversible terminator nucleotide, such as N6-(2-nitrobenzyl)-dAMP (Wu et al. Nucleic Acids Res. 2007;35(19):6339-49), which prevents elongation by polymerases until irradiation with UV light converts it back to the natural nucleotide dAMP. Alternately, if an incorporated photocleavable dNMP blocks the recognition or nicking activity of the nicking endonuclease in the isothermal amplification scheme described above, that would indirectly prevent amplification of molecules unless they are irradiated with UV light. A variation on this method is to introduce a photocleavable group in the backbone of the DNA replacing one or more bases of the recognition site of the nicking endonuclease such that phocleavage of that group, possibly followed by enzymatic treatment, generates a 3′ OH suitable for elongation by the strand-displacing polymerase, thus enabling the first strand-displacement amplification step and simultaneously generating a functional recognition site for the nicking endonuclease.

(3) In the third approach, the scheme for selectively amplifying the “correct” molecules is to selectively destroy the “incorrect” molecules, so that the only remaining amplifiable molecules are the correct ones. One method for rendering DNA molecules un-amplifiable is to expose them to a photoaffinity labeling reagent such as ethidium monoazide, which forms covalent adducts to DNA when irradiated with blue light (e.g. 458 nm), rendering them incapable of amplification by a polymerase (Takahashi et al. PLoS One. 2014 Feb. 5; 9(2):e82624.). In this system, ethidium monoazide is introduced to the reaction chamber and then tightly-focused blue laser light is used to destroy the “incorrect” molecules by only photo-activating the photoaffinity reagent at the locations where the “incorrect” molecules are anchored. When amplification buffer is subsequently introduced to the reaction chamber, only the “correct” molecules are amplified. (Several other photoactivatible DNA crosslinking reagents can also be used. Alternately, one can also directly destroy the “incorrect” DNA molecules using focused 260 nm light.) Finally, if the molecules are anchored via a photocleavable linker as described above, one one selectively release the every “incorrect” molecule and then thoroughly flush the reaction chamber so that only “correct” molecules remain when the amplification reagents are introduced.

7. “Multiple single molecules” reaction cycle images. One can track the construction of a single DNA molecule through several iterations of the reaction cycle using single-molecule fluorescence. These experiments are performed in a double-sided tape style sample chamber (Joo C, Ha T. Cold Spring Harbor Protocol 2012 Oct. 1; 2012(10):1104-8.) constructed from a PEG/PEG-biotin (99:1) functionalized no. 1.5 coverslip (Microsurfaces, Inc., Englewood, N.J.) and a laser-machined 3×1 in. piece of ⅛ in. clear acrylic with two holes that serve as input and output ports for reagents, (analogous to the drilled microscope slide described in Joo et al.) The sample chamber is mounted on a custom-built TIRF microscope based on an Olympus IX83 microscope with a mechanized staged with Z-drift compensation and imaged via a 100x oil-immersion TIRF objective. The sample chamber is first filled with 20 μL of wash buffer (WB; 50 mM KCl, 50 mM Tris HCl pH 8.0, 0.2 mg/mL BSA (NEB)) to wet it. Subsequently, the sample chamber is filled with a ˜5 pM solution of 0.04 μm FluoSpheres NeutrAvidin-labeled yellow-green fluorescent microspheres (Life Technologies) in WB for 1 minute and then washed twice with 50 μL of WB (1 wash=50 μL), to generate a very sparse peppering of fiducial markers useful for image registration and for focusing the microscope. The sample chamber is then filled with a 0.2 mg/mL NeutrAvidin (Life Technologies) solution in WB for 1 minute, followed by washing twice with WB, to coat the PEG-biotin surface with NeutrAvidin molecules for subsequent starter DNA immobilization. The sample chamber is then filled with imaging buffer (IB; WB with 2.5 mM protocatechuic acid (PCA), 1 mM 6-Hydroxy-2,5,7,8-tetramethylchroman-2-carboxylic acid (Trolox), and 5 nM protocatechuic acid dioxygenase (PCD)) and then imaged three times to record background fluorescence profiles of the Cy3 and Cy5 channels. Cy3 fluorescence is excited in TIRF mode via a 532 nm diode laser and Cy5 fluorescence is excited with a co-aligned 640 nm diode laser (Blue Sky Research, Milpitas, Calif.). All images are collected on an Andor iXon EMCCD camera (Andor Technology Ltd., Belfast, UK) with 512×512 resolution, 200 ms exposure, and EM gain 6.

Blunt starter dsDNA comprising of a 43 nt + strand with 5′ biotin group and a − strand with a 5′ PO₄ and a 3′ Alexa Fluor 647-label (synthesized by IDT) is immobilized on the sample chamber surface by filling the chamber with a 50 pM solution of starter DNA in WB for 1 minute and then washing twice with WB.

Starter DNA molecule:

```
SEQ ID NO: 27
5′ {Biotin}-TTATAGATATCGTATCAGCTGCTGGACATTTAGGACCGTACCG-OH 3′
                          ||||||||||||||||||||||||||||||||||||||||||
  3′ {AF 647}-ATATCTATAGCATAGTCGACGACCTGTAAATCCTGGCATGGC-PO₄ 5′
SEQ ID NO: 28
```

The sample chamber is then imaged for Cy3 and Cy5 channel fluorescence, which showed hundreds of discrete spots corresponding to individual starter DNA molecules. (Single-molecule fluorescence is confirmed with these imaging parameters by observation of single-step photobleaching of spots.) The reaction cycle is then carried out for three complete iterations, taking Cy3 and Cy5 channel fluorescence images after each step with the sample chamber filled with IB, and without moving the microscope stage. Ligation steps are performed by filling the reaction chamber with a Quick Ligation reaction mixture containing 250 nM iCy3 GT ExU (described above) for 2 minutes and then washing twice with WB. Deprotection steps are performed by filling the reaction chamber with a reaction mixture containing 0.05 U/μL of USER Enzyme in 1x CutSmart buffer for 5 minutes and then washing with WB. Fill-in steps are performed by filling the reaction chamber with a reaction mixture containing 0.05 U/μL of Klenow and 0.33 μM dNTPs in 1× CutSmart buffer for 1 minute, and then washing with WB. All reactions are performed at room temperature, and all reagents except WB are stored on ice prior to use. Recorded images are processed using Fiji (Schindelin, J.; Arganda-Carreras, I. & Frise, E. et al. (2012), Nature methods 9(7): 676-682.) by subtracting the average of the three background images from each from reaction cycle image and then aligning the images using the StackReg plugin. The appearance of a spot in the Cy3 channel co-localized with a Cy5 spot indicates a successful ligation of an ExU molecule to an immobilized starter molecule, and subsequent disappearance of the Cy3-channel spot following incubation with the deprotection enzymes indicates successful deprotection of that molecule. (Fill-in steps do not cause a change in the fluorescence signal.) The cyclic appearance and disappearance of the Cy3-channel spot in the center of the highlighted region demonstrates that the indicated starter molecule has successfully completed three complete iterations of the reaction cycle.

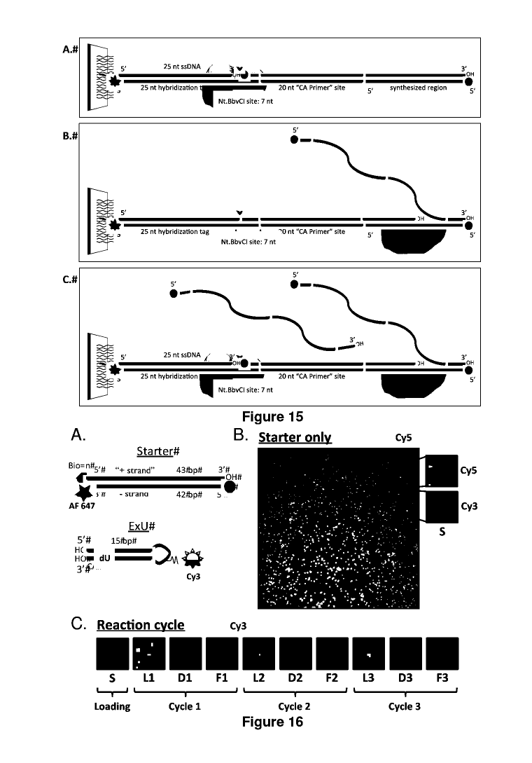

FIG. 16. Tracking a single starter DNA molecule through three iterations of the reaction cycle. Three iterations of the reaction cycle in a sample chamber mounted on a TIRF microscope have been demonstrated.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What we claim is:

1. A method comprising: (a) providing a reaction chamber comprising a solid support bound to a single starter double-stranded (ds) DNA molecule comprising a free end, (b) introducing one or more extension molecules and one or more enzymes capable of joining a payload region of an extension molecule to the free end of starter dsDNA molecule to the reaction chamber wherein the extension molecule comprises an cleavable linker, (c) removing unligated extension molecules and the one or more enzymes from the reaction chamber, (d) determining that the payload region of the extension molecule is joined to the free end of the starter dsDNA molecule resulting in an elongation of the free end of the starter DNA molecule, (e) optionally repeating steps (b) to (d) until the elongation is determined, (f) cleaving the cleavable linker of the extension molecule, (g) removing the non-payload region of the cleaved extension molecule from the reaction chamber, (h) determining that the non-payload region of the cleaved extension molecule is cleaved and removed from the reaction chamber, (i) optionally repeating steps (f) and (g) until the non-payload region is removed from the reaction chamber, and (i) optionally performing one or more cycles of steps (b) to (h) such that each cycle results in the extension of the starter dsDNA molecule with one or more payload regions. In some embodiments, the (b) introducing step comprises: (i) introducing a DNA polymerase and dNTP, (ii) optionally removing the DNA polymerase and dNTP from the reaction chamber, and (iii) introducing a extension molecule and a ligase.

2. A starter double-stranded (ds) DNA molecule comprising: (i) a starter solid support, and (ii) a + strand at least partially complementarily paired with a − strand, wherein both the + strand and the − strand each comprise a 5′ end and a 3′ end, optionally the − strand comprises an overhang of at least one nucleotide at the 5′ end of the − strand, and a PO₄ at the 5′ of the − strand, and the receptor is bound to the ligand, wherein the starter solid support is linked directly or indirectly to the 5′ end of the + strand.

3. An extension molecule comprising: (i) a + strand at least partially complementarily paired with a − strand, wherein both the + strand and the − strand each comprise a 5′ end and a 3′ end or the + strand and the − strand form a hairpin loop, (ii) optionally a cleavable linker in a region where the + strand and the − strand form a duplex, or a cleavable linker in the − strand, at the 3′ end of the + strand or the 5′ end of the − strand, and (iii) optionally a PO₄, labelling compound or ligand linked to the 5′ end of the − strand, the 3′ end of the + strand, or a loop of the hairpin loop.

4. The extension molecule of claim 3, wherein the payload region comprises polynucleotide from the 3′ end of the − strand to the 5′ end of the − strand, or where cleavage takes place from the cleaving of the cleavable linker, or polynucleotides of the − strand which are designed or configured to be joined or ligated to the 5′ end of the − strand of the starter dsDNA molecule.

5. The extension molecule of claim 3, comprising: (i) a + strand at least partially complementarily paired with a − strand, wherein both the + strand and the − strand each comprise a 5′ end and a 3′ end or the + strand and the − strand form a hairpin loop, (ii) a cleavable linker in a region where the + strand and the − strand form a double strand, or a cleavable linker in the − strand, and (iii) optionally a labelling compound or ligand linked to the 5′ end of the − strand or a loop of the hairpin loop.

6. The extension molecule of claim 3, comprising: (i) a + strand at least partially complementarily paired with a − strand, wherein both the + strand and the − strand each comprise a 5′ end and a 3′ end or the + strand and the − strand form a hairpin loop, (ii) a cleavable linker in a region where the + strand and the − strand form a double strand, or a cleavable linker in the − strand, and (iii) a labelling compound or ligand linked to the 5′ end of the − strand or a loop of the hairpin loop.

7. The extension molecule of claim 3, comprising: (i) a + strand at least partially complementarily paired with a − strand, wherein both the + strand and the − strand each comprise a 5′ end and a 3′ end, (ii) a cleavable linker in the − strand, and (iii) a labelling compound or ligand linked to the 5′ end of the − strand.

8. The extension molecule of claim 3, comprising: (i) a + strand at least partially, of fully, complementarily paired with a − strand, wherein both the + strand and the − strand each comprise a 5′ end and a 3′ end or the + strand and the − strand form a hairpin loop, (ii) a cleavable linker in a region where the + strand and the − strand form a double strand, and (iii) a labelling compound or ligand linked to the 5′ end of the − strand or a loop of the hairpin loop.

9. The extension molecule of claim 3, comprising: (i) a + strand at least partially complementarily paired with a − strand, wherein both the + strand and the − strand each comprise a 5′ end and a 3′ end or the + strand and the − strand form a hairpin loop, (ii) a cleavable linker in a region where the + strand and the − strand form a double strand, or a cleavable linker in the − strand or at the 5′ end of the + strand, and (iii) one or more labelling compound wherein each labelling compound is linked to a nucleotide within the constant region.

10. The extension molecule of claim 3, comprising: (i) a + strand at least partially, or fully, complementarily paired with a − strand, wherein both the + strand and the − strand each comprise a 5′ end and a 3′ end, (ii) optionally a cleavable linker at the 3′ end of the + strand or the 5′ end of the − strand, and (iii) a PO₄, labelling compound or ligand linked to the 5′ end of the − strand or the 3′ end of the + strand, wherein the payload region is the entire − strand. In some embodiments, the cleavable linker is located between the 5′ end of the − strand or the 3′ end of the + strand, and the labelling compound or ligand.

11. The extension molecule of claim 3, comprising: (i) a + strand at least partially, or fully, complementarily paired with a − strand, wherein both the + strand and the − strand each comprise a 5′ end and a 3′ end, (ii) a PO₄, a cleavable linker, and a labelling compound or ligand linked, in this 3′ to 5′ sequence, to the 5′ end of the − strand, wherein the payload region is the entire − strand.

12. The extension molecule of claim 3, comprising: (i) a + strand at least partially, or fully, complementarily paired with a − strand, wherein both the + strand and the − strand each comprise a 5′ end and a 3′ end, (ii) a PO₄ linked to the 5′ end of the − strand, and (iii) a cleavable linker, and a labelling compound or ligand linked, in this 5′ to 3′ sequence, to the 3′ end of the + strand, wherein the payload region is the entire − strand.

13. The extension molecule of claim 3, comprising: (i) a + strand at least partially, or fully, complementarily paired with a − strand, wherein both the + strand and the − strand each comprise a 5′ end and a 3′ end, (ii) a PO₄ linked to the 5′ end of the − strand, and (iii) a labelling compound or ligand linked to the 3′ end of the + strand, wherein the payload region is the entire − strand.
